(19) United States
(12) Patent Application Publication
(10) Pub. No.: US 2023/0298758 A1
(43) Pub. Date: Sep. 21, 2023

(54) Systems and Methods for Evaluating Gestational Progress and Applications Thereof

(71) Applicants: The Board of Trustees of the Leland Stanford Junior University, Stanford, CA (US); Statens Serum Institut, Copenhagen (DK); The Regents of the University of California, Oakland, CA (US)

(72) Inventors: Liang Liang, Palo Alto, CA (US); Michael P. Snyder, Stanford, CA (US); Mads Melbye, Vanlose (DK); Songjie Chen, Newark, CA (US); Larry Rand, San Francisco, CA (US); Laura Jelliffe-Pawlowski, Berkeley, CA (US); Xiaotao Shen, Sunnyvale, CA (US)

(73) Assignees: The Board of Trustees of the Leland Stanford Junior University, Stanford, CA (US); Statens Serum Institut, Copenhagen (DK); The Regents of the University of California, Oakland, CA (US)

(21) Appl. No.: 18/251,702

(22) PCT Filed: Nov. 8, 2021
(86) PCT No.: PCT/US21/72295

§ 371 Date: May 3, 2023

Related U.S. Application Data
(60) Provisional application No. 63/110,869, filed on Nov. 6, 2020.

(51) Int. Cl. G16H 50/20; A61B 10/00; A61B 8/00

(52) U.S. Cl. CPC G16H 50/20; A61B 10/007; A61B 8/4416

(57) ABSTRACT

Methods to compute gestational age and gestational health and applications thereof are described. Generally, systems utilize analyte measurements to determine a gestational age and gestational health, which can be used as a basis to perform interventions and treat individuals.

### CROSS REFERENCE TO RELATED APPLICATIONS

This application claims priority to U.S. Provisional Application Ser. No. 63/110,869, entitled “Methods for Evaluating Gestational Progress and Applications Thereof,” filed Nov. 6, 2020, which is incorporated herein by reference in its entirety.

### FIELD OF TECHNOLOGY

The disclosure is generally directed to processes to evaluate gestational progress and applications thereof, and more specifically to methods for evaluating gestational age, time to labor, preterm birth, and preterm abortion including diagnostics to be utilized for clinical interventions.

### BACKGROUND

Pregnancy is one of the most critical periods for mother and child. It involves a tremendous flow of physiological changes and metabolic adaptations week by week, and even small deviations from the norm may have detrimental consequences. There are 300,000 pregnancy and birth-related maternal deaths and 7.5 million perinatal deaths annually worldwide. In addition, 30% of all pregnancies end in miscarriage (<20 weeks), and preterm birth (<37 weeks). The latter is the leading cause of global neonatal morbidity and mortality and is observed for 7-17% of all pregnancies. With 170 million pregnancies yearly worldwide, even small improvements in obstetric health care, based on a better understanding of how pregnancy is regulated, may impact on the wellbeing of a large number of women and children.

Although ultrasound is used in clinics for estimating the gestational age, its accuracy is suboptimal with only 40% of the newborns delivered within 7 days of the predicted due dates. The accuracy is also decreased after the first trimester. Thus, there remains a need in the art for improved methods of estimating gestational age and predicting time to delivery and labor onset.

### SUMMARY

Several embodiments are directed toward determining gestational progress and/or gestational health of an individual. In many embodiments, a urine sample is collected from a pregnant individual and analytes from the urine sample are measured. In several embodiments, a predictive computational model is constructed and trained to predict gestational progress and/or gestational model. In many embodiments, analytes measurements of a pregnant individual are utilized within a constructed and trained computational model to predict gestational progress and/or gestational health. In several embodiments, the predicted gestational progress and/or gestational health is utilized to perform a clinical intervention or treat the individual.

In an embodiment, gestational age or time-to-delivery of an individual is determined. Measurements of one or more analytes is obtained, the analytes are derived from one or more urine sample collected from an individual to be assessed. Using a predictive computational model and the measurements of the one or more analytes, a gestational age or a time to delivery of the individual is predicted.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a flow chart for determining gestational progress or gestational health in accordance with various embodiments.

FIG. 2 provides a flow chart for constructing and training a computational model to determine a pregnant individual's gestational progress and/or gestational health in accordance with various embodiments.

FIG. 3 provides a flow chart for utilizing a computational model to determine gestational progress and/or gestational health in accordance with various embodiments.

FIG. 4 provides a schematic of the sampling time points for individual participants, utilized in accordance with various embodiments. Each row represents an individual participant. The histogram and bar on the top and the right show the number of samples collected at each gestational age range (bin width=0.5 weeks) and from each individual participant, respectively. Dots represent samples taken during pregnancy or after childbirth, and triangles represent childbirth.

FIG. 5 principal component analysis (PCA) on quality of urine metabolic data, generated in accordance with various embodiments.

FIG. 6 provides principal component analysis (PCA) distributed individual urine samples according to gestational age (based on metabolic peaks with QC RSD<30%), generated in accordance with various embodiments. The two PCs explaining the largest part of the variation are shown.

FIG. 7 provides a volcano plot showing altered metabolic peaks during pregnancy, using the linear regression model (FDR adjusted P-value<0.05) and SAM test (FDR adjusted P-value<0.05), generated in accordance with various embodiments. Dots on right represent metabolic features that increased during pregnancy and dots on left represent features that decreased during pregnancy.

FIG. 8 provides a data graph showing the importance of 28 metabolic peaks that were utilized as features, which were selected based on the Boruta algorithm, in a gestational age prediction model in accordance with various embodiments.

FIGS. 9 and 10 provide data graphs depicting the ability of a gestational age prediction model utilizing 28 metabolic peaks as features, which were selected based on the Boruta algorithm, in an internal validation data set (FIG. 13) and an external validation data set (FIG. 14), generated in accordance with various embodiments.

FIG. 11 provides a data graph showing the importance of 21 metabolites that were utilized as features in a gestational age prediction model in accordance with various embodiments.

FIG. 12 provides a pie chart depicting the importance ratio of different chemical classes in gestational age prediction model, generated in accordance with various embodiments.

FIGS. 13 and 14 provide data graphs depicting the ability of a gestational age prediction model utilizing 21 metabolites as features in an internal validation data set (FIG. 13) and an external validation data set (FIG. 14), generated in accordance with various embodiments.

FIG. 15 provides data graphs depicting the gestational age prediction accuracy for individual participants, generated in accordance with various embodiments.

FIG. 16 provides a Venn diagram depicting the overlap between the metabolites in the prediction model for gestational age and the time-to-delivery model, generated in accordance with various embodiments.

FIG. 17 provides a data graph showing the importance of 21 metabolites that were utilized as features in a time-to-delivery prediction model in accordance with various embodiments.

FIGS. 18 and 19 provide data graphs depicting the ability of a time-to-delivery prediction model utilizing 21 metabolites as features in an internal validation data set (FIG. 18) and an external validation data set (FIG. 19), generated in accordance with various embodiments.

FIG. 20 provides data graphs depicting the time-to-delivery prediction accuracy for individual participants, generated in accordance with various embodiments.

FIG. 21 provides a cluster map showing the clustering of identified metabolites markers for gestational age prediction models, generated in accordance with various embodiments. Based on different stages of gestational age (Y-axis, showing gestational weeks), markers were clustered into two main groups, one was upregulated in early stages and downregulated in late stages, while the other group showed a contrast pattern, with an upregulation in late stages.

FIG. 22 provides data graphs of fuzzy-c mean clustering of metabolite biomarkers based on gestational weeks, generated in accordance with various embodiments. The identified metabolite markers could be clustered into two groups, one with a consistent downregulation as pregnancy progresses followed by a return to normal levels postpartum.

FIG. 23 provides data graphs of five metabolite markers that decrease during pregnancy and increase after childbirth, generated in accordance with various embodiments.

FIG. 24 provides data graphs of nineteen metabolite markers that increase during pregnancy and decrease after childbirth, generated in accordance with various embodiments.

### DETAILED DESCRIPTION

Turning now to the drawings and data, methods to determine gestational progress and/or gestational health based on analyte measurements derived from a pregnant individual and applications thereof in accordance with various embodiments are described. In some embodiments, a urine sample is collected from a pregnant individual and analytes in the sample are measured. In some embodiments, a panel of analyte measurements are used to compute gestational progress (e.g., gestational age and/or time to delivery) and provide an indication of an individual's pregnancy timeline. In some embodiments, a panel of analyte measurements are used to compute an indication of a pregnancy health including various complications, such as spontaneous abortion. Many embodiments utilize an individual's gestational age and/or health determination to perform further diagnostic testing and/or treat the individual. In some instances, a diagnostic can include medical imaging (e.g., ultrasonography), periodic medical checkups, fetal monitoring, blood tests (e.g., glucose), microbial culture tests, genetic screening, chorionic villus sampling, and amniocentesis. In some instances, a treatment can include a medication, a dietary supplement, Caesarian delivery, a surgical procedure, and any combination thereof.

Many treatment regimens and clinical decisions in obstetrics depend on an accurate estimation of the timing and progression of pregnancy. Current clinical determination of gestational age and due date are typically based on information about last menstruation date or ultrasound imaging, which can be imprecise. An accurate and cost-effective method for estimating gestational age and delivery time is in need.

The present disclosure is based on the discovery of analyte biomarkers that are within urine that can be used in monitoring women during pregnancy to determine gestational age, time until delivery, indicate preterm labor, and diagnose spontaneous abortion. Untargeted analyte investigations were performed on urine samples from cohorts of pregnant women (see attached manuscript and figures). These studies revealed analyte alterations in urine during normal pregnancy. Many analyte measurements and the dynamics of the various analytes were shown to be timed precisely according to pregnancy progression and can be used to assess gestational progress, preterm labor and spontaneous abortion. In various embodiments, computational models utilize analyte measurements derived from urine to determine gestational progress and health.

### Analytes Indicative of Gestational Progress and Health

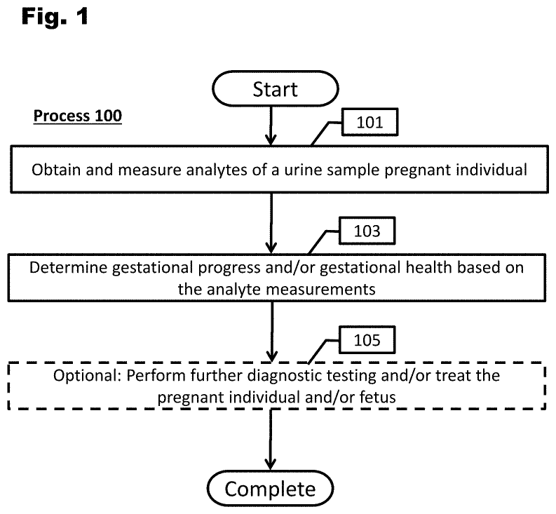

A process for determining pregnancy progress, gestational age, time to delivery, and/or a gestational health using analyte measurements derived from urine, in accordance with various embodiments, is shown in FIG. 1. This embodiment is directed to determining an indication of gestational progress and/or health of an individual and applies the knowledge garnered to perform further diagnostics and/or treat an individual. For example, this process can be used to identify an individual having a particular analyte constituency that is indicative of spontaneous abortion and treat that individual with estrogen and/or progesterone and further monitor the individual (e.g., weekly medical checkups).

In a number of embodiments, analytes and analyte measurements are to be interpreted broadly as clinical and molecular constituents and measurements that can be captured in medical and/or laboratory setting and are to include metabolites, protein constituents, genomic DNA, transcript expression, and lipids. In some embodiments, metabolites are to include intermediates and products of metabolism such as (for example) sugars, amino acids, nucleotides, antioxidants, organic acids, polyols, vitamins, and the like. In various embodiments, protein constituents are chains of amino acids which are to include (but not limited to) peptides, enzymes, receptors, ligands, antibodies, transcription factors, cytokines, hormones, growth factors and the like. In some embodiments, genomic DNA is DNA of an individual and includes (but is not limited to) copy number variant data, single nucleotide variant data, polymorphism data, mutation analysis, insertions, deletions, epigenetic data and partial and full genomes. In various embodiments, transcript expression is the evidence of RNA molecules of a particular gene or other RNA transcripts, and is to include (but is not limited to) analysis of expression levels of particular transcript targets, splicing variants, a class or pathway of gene targets, and partial and full transcriptomes. In some embodiments, lipids are a broad class of molecules that include (but are not limited to) fatty acid molecules, fat soluble vitamins, glycerolipids, phospholipids, sterols, sphingolipids, prenols, saccharolipids, polyketides, and the like.

In some embodiments, clinical data and/or personal data can be additionally used to indicate gestation age and/or health. In some embodiments, clinical data is to include medical patient data such as (for example) weight, height, heart rate, blood pressure, body mass index (BMI), clinical tests and the like. In various embodiments, personal data is to include data captured by an individual such as (for example) wearable data, physical activity, diet, substance abuse and the like.

Referring back to FIG. 1, process 100 begins with obtaining and measuring (101) analytes from a urine sample of a pregnant individual. In some embodiments, an individual's sample is collected during fasting, or in a controlled clinical assessment. A number of methods are known to collect urine samples from an individual and can be used within various embodiments. In several embodiments, analytes are collected over a period a time (e.g., across pregnancy timeline) and measured at each time point, resulting in a dynamic analysis of the analytes. In some of these embodiments, analytes are measured with periodicity (e.g., weekly, monthly, trimester).

In a number of embodiments, an individual is any individual that has their analytes extracted and measured, especially individuals that have an indication of pregnancy. In some embodiments, an individual has been diagnosed as being pregnant (e.g., as determined by urine test or ultrasound). Embodiments are also directed to an individual being one that has not yet been diagnosed as pregnant.

A number of analytes can be used to indicate gestation age and/or health, including (but not limited to) metabolites, protein constituents, genomic DNA, transcript expression, and lipids. In some embodiments, clinical data and/or personal data can be additionally used to indicate gestation age and/or health. Analytes can be detected and measured by a number of methods, including nucleic acid and protein sequencing, mass spectrometry, colorimetric analysis, immunodetection, and the like.

In several embodiments, analyte measurements are performed by taking a single time-point measurement. In many embodiments, the median and/or average of a plurality of time points for participants with multiple time-point measurements are utilized. Various embodiments incorporate correlations, which can be calculated by a number of methods, such as the Spearman correlation method. A number of embodiments utilize a computational model that incorporates analyte measurements, such as linear regression and elastic net models. Significance can be determined by calculating p-values and/or contribution (also referred to as importance), which may be corrected for multiple hypotheses testing. It should be noted however, that there are several correlation, computational models, and statistical methods that can utilize analyte measurements and may also fall within some embodiments of the invention.

In a number of embodiments, dynamic correlations use a ratio of analyte measurements between two time points, a percent change of analyte measurements over a period of time, a rate of change of analyte measurements over a period of time, or any combination thereof. Several other dynamic measurements may also be used in the alternative or in combination in accordance with multiple embodiments.

Using static and/or dynamic measurements of analytes, process 100 determines (103) gestational progress and/or gestational health based on the analyte measurements. In many embodiments, the correlations and/or computational models can be used to indicate gestational progress and/or gestational health. In several embodiments, determining analyte correlations or modeling gestational progress and/or gestational health is used to substitute other gestational tests, such as (for example) ultrasonography. In various embodiments, measurements of analytes can be used as a precursor indicator to determine whether to perform a further clinical test, such as (for example) ultrasonography.

Having determined an individual's gestational progress and/or gestational health, a further diagnostic test can optionally be performed or the pregnant individual and/or fetus can optionally be treated (105). In some instances, a diagnostic can include medical imaging (e.g., ultrasonography), periodic medical checkups, fetal monitoring, blood tests (e.g., glucose), microbial culture tests, genetic screening, chorionic villus sampling, amniocentesis, and any combination thereof. In some instances, a treatment can include a medication, a dietary supplement, Caesarian delivery, a surgical procedure, and any combination thereof.

While specific examples of determining an individual's gestational progress and/or gestational health are described above, one of ordinary skill in the art can appreciate that various steps of the process can be performed in different orders and that certain steps may be optional according to some embodiments. As such, it should be clear that the various steps of the process could be used as appropriate to the requirements of specific applications. Furthermore, any of a variety of processes for determining an individual's gestational progress and/or gestational health appropriate to the requirements of a given application can be utilized in accordance with various embodiments.

Modeling Gestational Progress and Health with Analyte Measurements

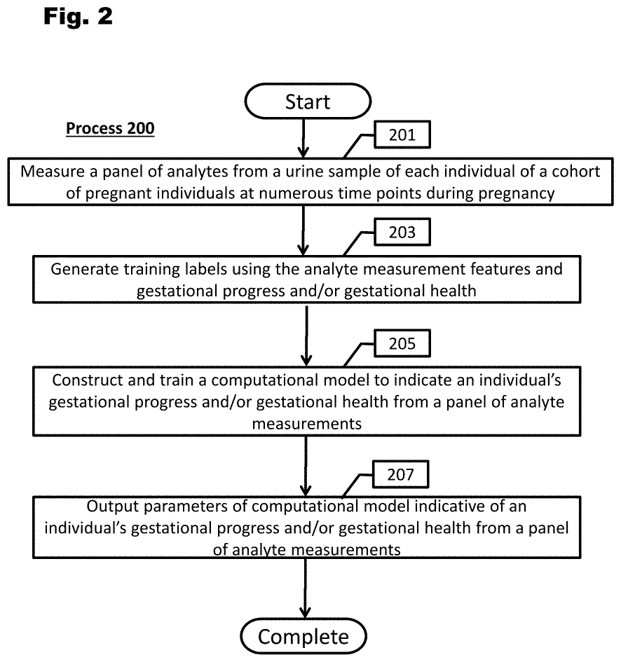

A process for constructing and training a computational model to indicate gestational progress and/or gestational health utilizing analyte measurements from a urine sample of pregnant individual, in accordance with various embodiments, is shown in FIG. 2. Process 200 measures (201) one or more analytes from one or more urine samples of each individual of a cohort of pregnant individuals. In some embodiments, an individual's urine sample is collected during fasting. A number of methods are known to collect urine samples from an individual and can be used within various embodiments of the invention.

In several embodiments, analytes are collected with periodicity across the timeline of pregnancy and postpartum. Accordingly, in some embodiments, analyte measurements are performed weekly, bi-weekly, monthly, per trimester, pre- and post-health event, after delivery, and any combination thereof. The precise extraction timeline will depend on the data to be collected and the model to be constructed In some embodiments, a plurality of urine samples is collected, the plurality of urine samples collected over a plurality of times during pregnancy. In some embodiments, at least two urine samples are collected at two individual timepoints. In some embodiments, at least three urine samples are collected at three individual time points. In some embodiments, at least one urine sample is collected in each trimester. In some embodiments, a urine sample is collected at a routine prenatal checkup, which typically occurs every four weeks between gestational ages 4 to 28 weeks, every two weeks between gestational ages 28 to 36 weeks, and every week between gestational ages 36 to 40 weeks. It should be understood, however, various factors such as the age of the pregnant individual or pre-exiting health problems, will influence the regularity of prenatal checkups.

A number of analytes can be used to determine gestational progress and/or gestational health, including (but not limited to) metabolites, protein constituents, genomic DNA, transcript expression, and lipids. In some embodiments, clinical data and/or personal data can be additionally used to determine gestational progress and/or gestational health. Analytes can be detected and measured by a number of methods, including nucleic acid and protein sequencing, mass spectrometry, colorimetric analysis, immunodetection, and the like. It should be noted that static, median, average, and/or dynamic analyte measurements can be used in accordance with various embodiments of the invention.

A cohort of pregnant individuals, in accordance with many embodiments, is a group of pregnant individuals that have had urine samples collected and analytes measured so that their data can be used to construct and train a computational model. A cohort will typically include individuals that are diagnosed as pregnant such that their analytes can be extracted along the pregnancy timeline. The number of individuals in a cohort can vary, and in some embodiments, having a greater number of individuals will increase the prediction power of a trained computer model. The precise number and composition of individuals will vary, depending on the model to be constructed and trained.

Using the analyte measurements and gestational progress and/or gestational health, process 200 generates (203) training labels that provide a correspondence between analyte measurement features and gestational progress and/or gestational health. In several embodiments, analyte measurements used to generate training labels are determinative of gestational progress and/or gestational health. In some embodiments, analyte measurements are standardized.

Based on studies performed, it has been found that several analyte measurements provide robust predictive ability, including (but not limited to) metabolites, protein constituents, genomic DNA, transcript expression, and lipids. A number of methods can be used to select analyte measurements to be used as features in the training model. In some embodiments, correlation measurements between analyte measurements and gestational progress and/or gestational health are used to select features. In various embodiments, a computational model is used to determine which analyte measurements are best predictors. For example, a linear regression model (e.g., LASSO), random forest model, or elastic net model can be used to determine which analyte measurement features provide the best predictive power as determined by their contribution.

A selection of predictive analyte measurement features is described in the Exemplary Embodiments. For instance, it has been found that the following 21 metabolites provide predictive power and one or more of these metabolites can be utilized as features within a model to predict predictive gestational age: 11α-hydroxyprogesterone β-D-glucuronide; ubiquinone (Q2); omega-3 arachidonic acid methyl ester; 5α-pregnane-3,20-dione; 5 β-pregnane-3α, 17-diol-20-one; pregnenolone; tetrahydrocorticosterone; progesterone; 21-hydroxypregnenolone; 5(Z),8(Z),11(Z)-eicosatrienoic acid methyl ester; cortisol; 3-acetoxypyridine; N-acetylmannosamine; N-(4-chlorophenyl)-4-piperidinamine; N-acetyllactosamine; propionyl-carnitine; N-acetylneuraminic acid; (2R)-3-hydroxyisovaleroylcarnitine; cAMP; thymine; and 4-hydroxycinnamic acid. Based on the foregoing, it should be understood that a number of combinations of analyte features can be used solitarily or combined in any fashion to be used to train a predictive computational model. In some embodiments, features of a gestational age prediction model includes measurements of at least two of the listed metabolites. In some embodiments, features of a gestational age prediction model includes measurements of at least three of the listed metabolites. In some embodiments, features of a gestational age prediction model includes measurements of at least four of the listed metabolites. In some embodiments, features of a gestational age prediction model includes measurements of at least five of the listed metabolites. In some embodiments, features of a gestation age prediction model includes measurements of at least six of the listed metabolites. In some embodiments, features of a gestation age prediction model includes at least measurements of seven of the listed metabolites. In some embodiments, features of a gestation age prediction model includes measurements of at least eight of the listed metabolites. In some embodiments, features of a gestation age prediction model includes measurements of at least nine of the listed metabolites. In some embodiments, features of a gestation age prediction model includes measurements of at least 10 of the listed metabolites. In some embodiments, features of a gestation age prediction model includes measurements of at least 15 of the listed metabolites. In some embodiments, features of a gestation age prediction model includes measurements of at least 20 of the listed metabolites. In some embodiments, features of a gestation age prediction model includes measurements of at least 21 of the listed metabolites.

It has also been found that the following 21 metabolites provide predictive power and one or more of these metabolites can be utilized as features within a model to predict time to delivery: 11α-hydroxyprogesterone β-D-glucuronide; 5 β-pregnane-3α, 17-diol-20-one; omega-3 arachidonic acid methyl ester; ubiquinone (Q2); tetrahydrocorticosterone; 5α-pregnane-3,20-dione; 5(Z),8(Z),11(Z)-eicosatrienoic acid methyl ester; 21-hydroxypregnenolone; cortisol; pregnenolone; 19-hydroxytestosterone; progesterone; propionyl-carnitine; androstane-3,17-diol; N-acetyllactosamine; N-(4-chlorophenyl)-4-piperidinamine; 3-acetoxypyridine; N-acetylneuraminic acid; N-acetylmannosamine; thymine; and deoxycytidine. Based on the foregoing, it should be understood that a number of combinations of analyte features can be used solitarily or combined in any fashion to be used to train a predictive computational model. In some embodiments, features of a time-to-delivery model includes measurements of at least two of the listed metabolites. In some embodiments, features of a time-to-delivery prediction model includes measurements of at least three of the listed metabolites. In some embodiments, features of a time-to-delivery prediction model includes measurements of at least four of the listed metabolites. In some embodiments, features of a time-to-delivery prediction model includes measurements of at least five of the listed metabolites. In some embodiments, features of a time-to-delivery prediction model includes measurements of at least six of the listed metabolites. In some embodiments, features of a time-to-delivery prediction model includes at least measurements of seven of the listed metabolites. In some embodiments, features of a time-to-delivery prediction model includes measurements of at least eight of the listed metabolites. In some embodiments, features of a time-to-delivery prediction model includes measurements of at least nine of the listed metabolites. In some embodiments, features of a time-to-delivery prediction model includes measurements of at least 10 of the listed metabolites. In some embodiments, features of a time-to-delivery prediction model includes measurements of at least 15 of the listed metabolites. In some embodiments, features of a time-to-delivery prediction model includes measurements of at least 20 of the listed metabolites. In some embodiments, features of a time-to-delivery prediction model includes measurements of at least 21 of the listed metabolites.

Training labels associating analyte measurement features and gestational progress and/or gestational health are used to construct and train (205) a computational model to determine an individual's gestational progress and/or gestational health. Various embodiments construct and train a model to determine the individual's pregnancy progression, time to delivery, and/or experiencing spontaneous abortion. A number of models can be used in accordance with various embodiments, including (but not limited to) ridge regression, K-nearest neighbors, LASSO regression, elastic net, least angle regression (LAR), random forest, and principal components analysis.

In several embodiments, computational models are built for dynamic observation. Accordingly, some embodiments of models incorporate analyte data of individuals at multiple time points across a pregnancy timeline such that the model can determine gestational progress across a pregnancy timeline selected. In some embodiments of models, a timeline is a full gestational timeline (i.e., from first missed menstruation or fertilization to birth) or a partial gestational timeline (e.g., first trimester, second trimester, third trimester). Various embodiments include postpartum analyte data and thus a timeline would include postpartum periods as well. It should be understood that any appropriate time period can be utilized in accordance with various embodiments of the invention.

In several embodiments, computational models can be built for static observation. Accordingly, some embodiments of models incorporate analyte data of individuals at a particular time point (or particular time points) of a pregnancy timeline (e.g., 4 weeks, 6 weeks, 8 weeks, 10 weeks, 12 weeks 16 weeks, 24 weeks, 28 weeks, 32 weeks, 36 weeks or 40 weeks). In some embodiments of models, a time point to be analyzed is related to time to birth (e.g., 1 week, 2 weeks, 3 weeks, 4 weeks, 6 weeks, or 8 weeks to birth). In some embodiments, a model incorporates analyte data related to a gestational event, especially events related to gestational health. Gestational events that can be modeled include delivery, spontaneous abortion, postpartum depression, gestational diabetes, gestational hypertension, gestational trophoblastic disease, preeclampsia, hyperemesis gravidarum (i.e., morning sickness), preterm labor or any other event that is related to gestation.

Models and sets of training labels used to train a model can be evaluated for their ability to accurately determine gestational progress and/or gestational health. By evaluating models, predictive abilities of analyte measurements can be confirmed. In some embodiments, a portion of the cohort data is withheld to test the model to determine its efficiency and accuracy. A number of accuracy evaluations can be performed, including (but not limited to) area under the receiver operating characteristics (AUROC), R-square error analysis, and mean square error analysis. In some embodiments, the contribution of each feature to the ability to predict outcome is determined. In some embodiments, top contributing features are utilized to construct the model. Accordingly, an optimized model can be identified.

Process 200 also outputs (207) the parameters of a computational model indicative of an individual's gestational age and/or gestational health from a panel of analyte measurements. Computational models can be used to determine an individual's gestational progress and/or gestational health, provide diagnoses, and treat an individual accordingly, as will be described in detail below.

While specific examples of processes for constructing and training a computational model to determine an individual's gestational progress and/or gestational health are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for constructing and training a computational model appropriate to the requirements of a given application can be utilized in accordance with various embodiments of the invention.

### Determination of an Individual's Pregnancy Progression and Potential Complications Using Analyte Measurements

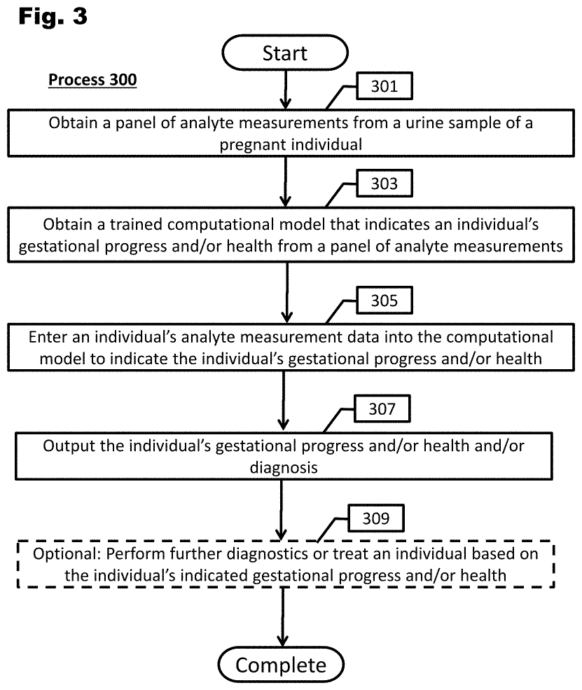

Once a computational model has been constructed and trained, it can be used to compute a determination of an individual's gestational progress and/or gestational health. As shown in FIG. 3, a method to determine an individual's gestational progress and/or gestational health using analyte measurements from the individual's urine sample and a trained computational model is provided in accordance with an embodiment of the invention. Process 300 obtains (301) a panel of analyte measurements from a urine sample of a pregnant individual.

In some embodiments, an individual's sample is collected during fasting. A number of methods are known to collect a sample from an individual and can be used within various embodiments of the invention. In several embodiments, analytes are collected and measured at numerous time points, resulting in a dynamic analysis of the analytes. In some of these embodiments, analytes are measured with periodicity (e.g., weekly, monthly, trimester).

A number of analytes can be used to determine gestational progress and/or gestational health, including (but not limited to) metabolites, protein constituents, genomic DNA, transcript expression, and lipids. In some embodiments, clinical data and/or personal data can be additionally used to determine gestational progress and/or gestational health. Analytes can be detected and measured by a number of methods, including nucleic acid and protein sequencing, mass spectrometry, colorimetric analysis, immunodetection, and the like. It should be noted that static, median, average, and/or dynamic analyte measurements can be used in accordance with various embodiments of the invention. In many embodiments, the precise panel of analytes to be measured depends on the constructed and trained computational model to be used, as the input analyte measurement data that will be needed to at least partially overlap with the features used to train the model. That is, there should be enough overlap between the feature measurements used to train the model and the individual's analyte measurements obtained such that gestational progress and/or gestational health can be determined.

In numerous embodiments, an individual has been diagnosed as being pregnant, as determined by any appropriate method (e.g., ultrasonography or urine test). Embodiments are also directed to an individual being one that has not been diagnosed as pregnant, especially in situations in which the individual is unaware of her pregnancy.

Process 300 also obtains (303) a trained computational model that indicates an individual's gestational progress and/or gestational health from a panel of analyte measurements. Any computational model that can compute an indicator of an individual's gestational progress and/or gestational health from a panel of analyte measurements can be used. In some embodiments, the computational model is constructed and trained as described in FIG. 2. The computational model, in accordance with various embodiments, has been optimized to accurately and efficiently indicate gestational progress and/or gestational health.

A number of models can be used in accordance with various embodiments, including (but not limited to) ridge regression, K-nearest neighbors, LASSO regression, elastic net, least angle regression (LAR), random forest, and principal components analysis.

Process 300 also enters (305) an individual's analyte measurement data into a computational model to indicate the individual's gestational progress and/or gestational health. In some embodiments, the analyte measurement data is used to compute an individual's gestational progress and/or gestational health in lieu of performing a traditional gestational analysis (e.g., ultrasonography). Various embodiments utilize the analyte measurement data and computational model in combination with a clinical diagnostic method.

Based on studies performed, it has been found that several analyte measurements provide robust predictive ability, including (but not limited to) particular metabolites, protein constituents, genomic DNA, transcript expression, and lipids. A number of methods can be used to select analyte measurements to be used as features in the training model. In some embodiments, correlation measurements between analyte measurements and gestational progress and/or gestational health are used to select features. In various embodiments, a computational model is used to determine which analyte measurements are best predictors. For example, a linear regression model (e.g., LASSO), random forest model, or elastic net model can be used to determine which analyte measurement features provide the best predictive power as determined by their contribution.

A selection of predictive analyte measurement features is described in the Exemplary Embodiments. It has been found that the following 21 metabolites provide predictive power and one or more metabolite measurements can be utilized as features within a model to predict predictive gestational age: 11α-hydroxyprogesterone β-D-glucuronide; ubiquinone (Q2); omega-3 arachidonic acid methyl ester; 5α-pregnane-3,20-dione; 5 β-pregnane-3a, 17-diol-20-one; pregnenolone; tetrahydrocorticosterone; progesterone; 21-hydroxypregnenolone; 5(Z),8(Z),11(Z)-eicosatrienoic acid methyl ester; cortisol; 3-acetoxypyridine; N-acetylmannosamine; N-(4-chlorophenyl)-4-piperidinamine; N-acetyllactosamine; propionyl-carnitine; N-acetylneuraminic acid; (2R)-3-hydroxyisovaleroylcarnitine; cAMP; thymine; 4-hydroxycinnamic acid; 2-[[(6E,8E)-1,12-diamino-11-(carboxymethylamino)-2,11-dihydroxy-3,10-dioxododeca-6,8-dien-2-yl]amino]acetic acid; tetrahydroaldosterone-3-glucuronide, β-casomorphin (1-4); Gly-Arg-Gly-Glu-Ser-Pro, retinol; androstane-3,17-diol; 1-oleoyl-2-hydroxy-Sn-glycero-3-phospho-(1′rac-glycerol); estrone glucuronide; pinolenic acid ethyl ester; N,N,N′,N′-tetrakis(2-hydroxyethyl)hexanediamide; and 1-(5-Fluoropentyl)-N-(naphthalen-2-yl)-1H-indole-3-carboxamide. Based on the foregoing, it should be understood that a number of combinations of analyte features can be used solitarily or combined in any fashion to be used to train a predictive computational model. In some embodiments, features of a gestational age prediction model includes measurements of at least two of the listed metabolites. In some embodiments, features of a gestational age prediction model includes measurements of at least three of the listed metabolites. In some embodiments, features of a gestational age prediction model includes measurements of at least four of the listed metabolites. In some embodiments, features of a gestational age prediction model includes measurements of at least five of the listed metabolites. In some embodiments, features of a gestation age prediction model includes measurements of at least six of the listed metabolites. In some embodiments, features of a gestation age prediction model includes at least measurements of seven of the listed metabolites. In some embodiments, features of a gestation age prediction model includes measurements of at least eight of the listed metabolites. In some embodiments, features of a gestation age prediction model includes measurements of at least nine of the listed metabolites. In some embodiments, features of a gestation age prediction model includes measurements of at least 10 of the listed metabolites. In some embodiments, features of a gestation age prediction model includes measurements of at least 15 of the listed metabolites. In some embodiments, features of a gestation age prediction model includes measurements of at least 20 of the listed metabolites. In some embodiments, features of a gestation age prediction model includes measurements of at least 21 of the listed metabolites.

It has also been found that the following 21 metabolites provide predictive power and one or more metabolite measurements can be utilized as features within a model to predict time to delivery: 11α-hydroxyprogesterone β-D-glucuronide; 5 β-pregnane-3α, 17-diol-20-one; omega-3 arachidonic acid methyl ester; ubiquinone (Q2); tetrahydrocorticosterone; 5α-pregnane-3,20-dione; 5(Z),8(Z),11(Z)-eicosatrienoic acid methyl ester; 21-hydroxypregnenolone; cortisol; pregnenolone; 19-hydroxytestosterone; progesterone; propionyl-carnitine; androstane-3,17-diol; N-acetyllactosamine; N-(4-chlorophenyl)-4-piperidinamine; 3-acetoxypyridine; N-acetylneuraminic acid; N-acetylmannosamine; thymine; deoxycitidine; tetrahydroaldosterone-3-glucuronide; 2-[[(6E,8E)-1,12-diamino-11-(carboxymethylamino)-2,11-dihydroxy-3,10-dioxododeca-6,8-dien-2-yl]amino]acetic acid; retinol; 1-oleoyl-2-hydroxy-Sn-glycero-3-phospho-(1′rac-glycerol); estrone glucuronide; (5.)-Androst-2-En-17-One; N,N,N′,N′-tetrakis(2-hydroxyethyl)hexanediamide; pinolenic acid ethyl ester; 2-chloro-3-deazadenosine; and 4-androstene-11β, 17β-diol-3-one. Based on the foregoing, it should be understood that a number of combinations of analyte features can be used solitarily or combined in any fashion to be used to train a predictive computational model. In some embodiments, features of a time-to-delivery model includes measurements of at least two of the listed metabolites. In some embodiments, features of a time-to-delivery prediction model includes measurements of at least three of the listed metabolites. In some embodiments, features of a time-to-delivery prediction model includes measurements of at least four of the listed metabolites. In some embodiments, features of a time-to-delivery prediction model includes measurements of at least five of the listed metabolites. In some embodiments, features of a time-to-delivery prediction model includes measurements of at least six of the listed metabolites. In some embodiments, features of a time-to-delivery prediction model includes at least measurements of seven of the listed metabolites. In some embodiments, features of a time-to-delivery prediction model includes measurements of at least eight of the listed metabolites. In some embodiments, features of a time-to-delivery prediction model includes measurements of at least nine of the listed metabolites. In some embodiments, features of a time-to-delivery prediction model includes measurements of at least 10 of the listed metabolites. In some embodiments, features of a time-to-delivery prediction model includes measurements of at least 15 of the listed metabolites. In some embodiments, features of a time-to-delivery prediction model includes measurements of at least 20 of the listed metabolites. In some embodiments, features of a time-to-delivery prediction model includes measurements of at least 21 of the listed metabolites.

Process 300 also outputs (307) a report containing an individual's gestational age, weeks to delivery, and/or gestational health result and/or diagnosis. Furthermore, based on an individual's indicated gestational progress and/or gestational health, the individual is optionally further examined and/or treated (309) to ameliorate a symptom related to the result and/or diagnosis. In several embodiments, an individual is provided with a personalized treatment plan. Further discussion of treatments that can be utilized in accordance with this embodiment are described in detail below, which may include various medications, dietary supplements, and surgical procedures.

While specific examples of processes for determining an individual's gestational progress and/or gestational health are described above, one of ordinary skill in the art can appreciate that various steps of the process can be performed in different orders and that certain steps may be optional according to some embodiments. As such, it should be clear that the various steps of the process could be used as appropriate to the requirements of specific applications. Furthermore, any of a variety of processes for computing an individual's gestational progress and/or gestational health appropriate to the requirements of a given application can be utilized in accordance with various embodiments.

### Feature Selection

As explained in the previous sections, analyte measurements are used as features to construct a computational model that is then used to indicate an individual's gestational progress and/or gestational health. Analyte measurement features used to train the model can be selected by a number of ways. In some embodiments, analyte measurement features are determined by which measurements provide strong correlation with gestational progress and/or gestational health. In various embodiments, analyte measurement features are determined using a computational model, such as Bayesian network, which can determine which analyte measurements influence or are influenced by an individual's gestational progress and/or gestational health. Embodiments also consider practical factors, such as (for example) the ease and/or cost of obtaining the analyte measurement, patient comfort when obtaining the analyte measurement, and current clinical protocols are also considered when selecting features.

Correlation analysis utilizes statistical methods to determine the strength of relationships between two measurements. Accordingly, a strength of relationship between an analyte measurement and gestational progress and/or gestational health can be determined. Many statistical methods are known to determine correlation strength (e.g., correlation coefficient), including linear association (Pearson correlation coefficient), Kendall rank correlation coefficient, and Spearman rank correlation coefficient. Analyte measurements that correlate strongly with gestational progress and/or gestational health can then be used as features to construct a computational model to determine an individual's gestational progress and/or gestational health.

In a number of embodiments, analyte measurement features are identified by a computational model, including (but not limited to) a Bayesian network model, LASSO, and elastic net. In some embodiments, the contribution of a feature to the predictive ability of the model is determined and features are selected based on their contribution. In some embodiments, the top contributing features are utilized. In some embodiments, the features that contribute over a percentage are selected (e.g., each feature that contributes at least 1% or the combination of top features that provide 90% contribution). In various embodiments, features that contribute at least 0.1%, 0.5%, 1%, 2%, 3%, 4%, 5%, or 10% to outcome prediction are selected. In various embodiments, the top features that in combination provide at least 50%, 75%, 80%, 90%, 95%, 99%, 99.5%, or 99.9% to outcome prediction are selected. In some embodiments, the Boruta algorithm is utilized to select analyte features (see Exemplary Embodiments for more details). The precise number of contributing features will depend on the results of the model and each feature's contribution. Various embodiments utilize an appropriate computational model that results in a number of features that is manageable. For instance, constructing predictive models from hundreds to thousands of analyte measurement features may have overfitting issues. Likewise, too few features can result in less prediction power.

### Biomarkers as Indicators of Gestation Age and Health

In several embodiments, biomarkers are detected and measured, and based on the ability to be detected and/or level of the biomarker, gestational progress and/or gestational health can be determined directly or via a computational model. Biomarkers that can be used in the practice of the invention include (but are not limited to) metabolites, protein constituents, genomic DNA, transcript expression, and lipids. As discussed in the Exemplary Embodiments, a number of biomarkers have been found to be useful to determine gestational progress and/or gestational health, including (but not limited to) 11α-hydroxyprogesterone β-D-glucuronide; ubiquinone (Q2); omega-3 arachidonic acid methyl ester; 5α-pregnane-3,20-dione; 5 β-pregnane-3α, 17-diol-20-one; pregnenolone; tetrahydrocorticosterone; progesterone; 21-hydroxypregnenolone; 5(Z),8(Z),11(Z)-eicosatrienoic acid methyl ester; cortisol; 3-acetoxypyridine; N-acetylmannosamine; N-(4-chlorophenyl)-4-piperidinamine; N-acetyllactosamine; propionyl-carnitine; N-acetylneuraminic acid; (2R)-3-hydroxyisovaleroylcarnitine; cAMP; thymine; 4-hydroxycinnamic acid; 19-hydroxytestosterone, androstane-3,17-diol; deoxycitidine; 2-[[(6E,8E)-1,12-diamino-11-(carboxymethylamino)-2,11-dihydroxy-3,10-dioxododeca-6,8-dien-2-yl]amino]acetic acid; tetrahydroaldosterone-3-glucuronide, β-casmorphin (1-4); Gly-Arg-Gly-Glu-Ser-Pro, retinol; androstane-3,17-diol; 1-oleoyl-2-hydroxy-Sn-glycero-3-phospho-(1′rac-glycerol); estrone glucuronide; pinolenic acid ethyl ester; N,N,N′,N′-tetrakis(2-hydroxyethyl)hexanediamide; and 1-(5-Fluoropentyl)-N-(naphthalen-2-yl)-1H-indole-3-carboxamide.

### Detecting and Measuring Levels of Biomarkers

Analyte biomarkers in a urine sample can be determined by a number of suitable methods. Suitable methods include chromatography (e.g., high-performance liquid chromatography (HPLC), gas chromatography (GC), liquid chromatography (LC)), mass spectrometry (e.g., MS, MS-MS), NMR, enzymatic or biochemical reactions, immunoassay, and combinations thereof. For example, mass spectrometry can be combined with chromatographic methods, such as liquid chromatography (LC), gas chromatography (GC), or electrophoresis to separate the metabolite being measured from other components in the biological sample. See, e.g., Hyotylainen (2012) Expert Rev. Mol. Diagn. 12(5):527-538; Beckonert et al. (2007) Nat. Protoc. 2(11):2692-2703; O'Connell (2012) Bioanalysis 4(4):431-451; and Eckhart et al. (2012) Clin. Transl. Sci. 5(3):285-288; the disclosures of which are herein incorporated by reference. Alternatively, analytes can be measured with biochemical or enzymatic assays. For example, glucose can be measured with a hexokinase-glucose-6-phosphate dehydrogenase coupled enzyme assay. In another example, biomarkers can be separated by chromatography and relative levels of a biomarker can be determined from analysis of a chromatogram by integration of the peak area for the eluted biomarker.

Immunoassays based on the use of antibodies that specifically recognize a biomarker may be used for measurement of biomarker levels. Such assays include (but are not limited to) enzyme-linked immunosorbent assay (ELISA), radioimmunoassays (RIA), “sandwich” immunoassays, fluorescent immunoassays, enzyme multiplied immunoassay technique (EMIT), capillary electrophoresis immunoassays (CEIA), immunoprecipitation assays, western blotting, immunohistochemistry (IHC), flow cytometry, and cytometry by time of flight (CyTOF).

Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A biomarker antigen can be used to immunize a mammal, such as a mouse, rat, rabbit, guinea pig, monkey, or human, to produce polyclonal antibodies. If desired, a biomarker antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface-active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a biomarker antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 31 42, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prev. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J Cancer 61, 497-501, 1995; Nicholls et al., J. Immunol. Meth. 165, 81-91, 1993).

Antibodies which specifically bind to a biomarker antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

Antibodies may be used in diagnostic assays to detect the presence or for quantification of the biomarkers in a biological sample. Such a diagnostic assay may comprise at least two steps; (i) contacting a biological sample with the antibody, wherein the sample is blood or plasma, a microchip (e.g., See Kraly et al. (2009) Anal Chim Acta 653(1):23-35), or a chromatography column with bound biomarkers, etc.; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem. 13:1014 (1974); Pain et al., J. Immunol. Methods 40:219 (1981); and Nygren, J. Histochem. and Cytochem. 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

In various embodiments, biomarkers in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate a two-dimensional array of spots for the biomarkers. See, e.g., Jungblut and Thiede, Mass Spectr. Rev. 16:145-162 (1997).

Two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in the one-dimensional array are further separated using a technique generally distinct from that used in the first separation step. For example, in the second dimension, biomarkers separated by isoelectric focusing are further resolved using a polyacrylamide gel by electrophoresis in the presence of sodium dodecyl sulfate (SDS-PAGE). SDS-PAGE allows further separation based on molecular mass. Typically, two-dimensional gel electrophoresis can separate chemically different biomarkers with molecular masses in the range from 1000-200,000 Da, even within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more biomarkers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a biomarker can be analyzed by gas phase ion spectrometry. In gas phase ion spectrometry, the spots can be analyzed using any suitable techniques, such as MALDI or SELDI.

In a number of embodiments, high performance liquid chromatography (H PLC) can be used to separate a mixture of biomarkers in a sample based on their different physical properties, such as polarity, charge and size. HPLC instruments typically consist of a reservoir, the mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

After preparation, biomarkers in a sample are typically captured on a substrate for detection. Traditional substrates include antibody-coated 96-well plates or nitrocellulose membranes that are subsequently probed for the presence of biomarkers. Alternatively, metabolite-binding molecules attached to microspheres, microparticles, microbeads, beads, or other particles can be used for capture and detection of biomarkers. The metabolite-binding molecules may be antibodies, peptides, peptoids, aptamers, small molecule ligands or other metabolite-binding capture agents attached to the surface of particles. Each metabolite-binding molecule may comprise a “unique detectable label,” which is uniquely coded such that it may be distinguished from other detectable labels attached to other metabolite-binding molecules to allow detection of biomarkers in multiplex assays. Examples include, but are not limited to, color-coded microspheres with known fluorescent light intensities (see e.g., microspheres with xMAP technology produced by Luminex (Austin, TX); microspheres containing quantum dot nanocrystals, for example, having different ratios and combinations of quantum dot colors (e.g., Qdot nanocrystals produced by Life Technologies (Carlsbad, CA); glass coated metal nanoparticles (see e.g., SERS nanotags produced by Nanoplex Technologies, Inc. (Mountain View, CA); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, CA); chemiluminescent dyes, combinations of dye compounds; and beads of detectably different sizes. See, e.g., U.S. Pat. Nos. 5,981,180, 7,445,844, 6,524,793, Rusling et al. (2010) Analyst 135(10): 2496-2511; Kingsmore (2006) Nat. Rev. Drug Discov. 5(4): 310-320, Proceedings Vol. 5705 Nanobiophotonics and Biomedical Applications II, Alexander N. Cartwright; Marek Osinski, Editors, pp. 114-122; Nanobiotechnology Protocols Methods in Molecular Biology, 2005, Volume 303; herein incorporated by reference in their entireties).

Mass spectrometry, and particularly SELDI mass spectrometry, is useful for detection of biomarkers. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting biomarkers. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

The methods for detecting biomarkers in a sample have many applications. For example, the biomarkers are useful in monitoring women during pregnancy, for example to determine gestational age, predict time until delivery, or assess risk of spontaneous abortion.

### Kits

In several embodiments, kits are utilized for monitoring women during pregnancy, wherein the kits can be used to detect analyte biomarkers as described herein. For example, the kits can be used to detect any one or more of the analyte biomarkers described herein, which can be used to determine gestational age, predict time until delivery, and/or assess risk of spontaneous abortion. The kit may include one or more agents for detection of one or more metabolite biomarkers, a container for holding a biological sample (e.g., urine) obtained from a subject; and printed instructions for reacting agents with the biological sample to detect the presence or amount of one or more biomarkers in the sample. The agents may be packaged in separate containers. The kit may further comprise one or more control reference samples and reagents for performing a biochemical assay, enzymatic assay, immunoassay, or chromatography. In various embodiments, a kit may include an antibody that specifically binds to a biomarker. In some embodiments, a kit may contain reagents for performing liquid chromatography (e.g., resin, solvent, and/or column).

A kit can include one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of monitoring women during pregnancy, e.g., to determine gestational age, predict time until delivery, and/or predict imminent spontaneous abortion.

### Applications and Treatments Related to Gestational Progress and Health

Various embodiments are directed to performing further diagnostics and or treatments based on a determination of gestational progress and/or gestational health. As described herein, a pregnant individual's gestational progress and/or gestational health is determined by various methods (e.g., computational methods, biomarkers). Based on one's gestational progress and/or gestational health, an individual can be subjected to further diagnostic testing and/or treated with various medications, dietary supplements, and surgical procedures.

### Clinical Diagnostics, Medications and Supplements

Several embodiments are directed to the use of medications and/or dietary supplements to treat an individual based on their gestational progress and/or gestational health determination. In some embodiments, medications and/or dietary supplements are administered in a therapeutically effective amount as part of a course of treatment. As used in this context, to “treat” means to ameliorate at least one symptom of the disorder to be treated or to provide a beneficial physiological effect. For example, one such amelioration of a symptom could be improvement in gestational health. Assessment of gestational progress and/or gestational health can be performed in many ways, including (but not limited to) the use of analyte measurements and sonography.

A therapeutically effective amount can be an amount sufficient to prevent reduce, ameliorate or eliminate the symptoms of diseases or pathological conditions susceptible to such treatment, such as, for example, spontaneous abortion or other gestational disorders. In some embodiments, a therapeutically effective amount is an amount sufficient to improve gestational health or reduce the risk of spontaneous abortion.

Various embodiments are directed towards getting an indication of gestational progress and performing an intervention and/or treatment thereupon. In some embodiments, when a pregnant individual is experiencing various symptoms at various points of gestational age or timeline to pregnancy (as determined by methods described herein), an intervention and/or treatment is performed. In some embodiments, treatments are performed when an individual exhibits symptoms that occur early and/or late according a determined gestational age or timeline to delivery. For example, a pregnant individual experiencing regular contractions prior to 37 weeks is considered to be in premature (preterm) labor, and a number of interventions and/or treatments can be performed. Likewise, gestation periods of longer than 42 weeks is considered to be a postterm pregnancy, additional monitoring, induction of labor, and/or Caesarian delivery is performed to avoid complications.

In a number of embodiments, when a pregnant individual is experiencing regular contractions, a gestational age can be determined, which would indicate whether the individual is experiencing preterm labor. In some embodiments, a gestational age is determined prior to any experienced contractions (e.g., as determined during the course of pregnancy) and based on the determined gestational age, an indication of preterm labor is determined. In accordance with various embodiments, it may be desirable to confirm that an individual is in preterm labor, and thus confirmation of labor can be performed by a number of means, including (but not limited to) cervical exam, sonography, testing for amniotic fluid, testing for fetal fibronectin, or any combination thereof. Treatments for preterm labor include (but not limited to) intravenous fluids, antibiotics (to treat infection), tocolytic medications (to slow or stop contractions), antenatal corticosteroids (to help mature fetus), cervical cerclage (to close up cervix), delivery of the baby, or any appropriate combination thereof. Tocolytic medications include (but not limited to) indomethacin, magnesium sulfate, orciprenaline, ritodrine, terbutaline, salbutamol, nifedipine, fenoterol, nylidrin, isoxsuprine, hexoprenaline, and atosiban. Antenatal corticosteroids include (but not limited to) dexamethasone and betamethasone. For more on treatment and care of preterm labor, see J. N. Robinson and E. R. Norwitz. Ed.: V. A. Barss. UpToDate, retrieved September 2019 (https://www.uptodate.com/contents/preterm-birth-risk-factors-interventions-for-risk-reduction-and-maternal-prognosis); C. J. Lockwood. Ed.: V. A. Barss. UpToDate, retrieved September 2019 (https://www.uptodate.com/contents/preterm-labor-clinical-findings-diagnostic-evaluation-and-initial-treatment); and H. N. Simhan and S. Caritis. Ed.: V. A. Barss. UpToDate, retrieved September 2019 (https://www.uptodate.com/contents/inhibition-of-acute-preterm-labor); the disclosure of which are each incorporated herein by reference).

In several embodiments, a pregnancy may go beyond a gestational age of 42 weeks, as determined by various methods described herein. As gestational age exceeds 42 weeks, the placenta may age, begin deteriorating, or fail. Accordingly, a number of embodiments are directed towards determining a gestational age and determine whether the individual is in a postterm pregnancy. In some embodiments, when a postterm pregnancy is indicated, additional monitoring can be performed, including (but not limited to) fetal movement recording (to monitor regular movements of fetus), doppler fetal monitor (to measure fetal heart rate), nonstress test (to monitor fetal heartbeat) and Doppler flow study (to monitor blood flow in and out of placenta). In some embodiments, when a postterm pregnancy is indicated, labor is induced and/or Caesarian delivery is performed.

In many embodiments, the gestational age and time to delivery are determined and used concurrently to determine whether an individual will experience preterm labor or a postterm pregnancy. In some embodiments, a time to delivery equal to or less than a gestational age of 37 weeks is determined, indicating that preterm labor is likely and thus interventions and treatments for preterm labor are performed. Likewise, in some embodiments, a time to delivery equal to or more than a gestational age of 42 weeks is determined, indicating that a postterm pregnancy is likely and thus monitoring, induced labor, or Casesarian delivery are performed.

In a similar manner, interventions and/or treatments can be performed at various other time points, as would be understood in the art. Accordingly, various methods described herein can determine gestational progress and based on symptoms, can perform an interventions and/or treatments. Critical time points include gestational ages of 20 weeks for determination of successful pregnancy and mitigating miscarriage, 24 weeks for determination age of viability, 28 weeks for determination of extreme preterm labor, 32 weeks for very preterm labor, 37 weeks for preterm labor, and 42 weeks for postterm pregnancy. At each time point, various interventions include prenatal checkups and monitoring, including measuring blood pressure, checking for urinary tract infection, checking for signs of preeclampsia, checking for signs of gestational hypertension, checking for signs of gestational diabetes, checking for signs of preterm labor, checking for signs of preterm rupture of membranes, measure heartbeat of fetus, measure fundal height, look for swelling in hands or feet, sampling for chorionic villus, check for risk of genetic disorders (e.g., Down syndrome and spina bifida), perform amniocentesis test, sonography, determine baby gender, and performing blood tests (e.g., glucose screening, anemia, status of Rh-positive or -negative).

A number of medications are available to treat spontaneous abortion and include (but are not limited to) estrogens, and progestogens (e.g., progesterone, dydrogesterone), or a combination thereof.

Numerous dietary supplements may also help to treat risk of spontaneous abortion. Various dietary supplements, such as folic acid, iron, calcium, vitamin D, docosahexaenoic acid (DHA), and iodine have been shown to have beneficial effects on pregnancy and reducing gestational disorders including spontaneous abortion. Thus, embodiments are directed to the use of dietary supplements, included those listed herein, to be used to treat an individual based on one's gestational progress and/or gestational health result.

### Exemplary Embodiments

Bioinformatic and biological data support the methods and systems of assessing gestational progress and applications thereof. In the attached manuscript and figures, exemplary methods and exemplary applications related to gestation that incorporate analyte panels, correlations, and computational models are provided.

### Longitudinal Urine Metabolic Profiling and Gestational Age Prediction in Pregnancy

The accurate dating of GA provides essential guidelines for the prenatal medical care. Current GA dating approaches based on the last menstrual period (LMP) are problematic given imprecise recollection of dates and symptoms like breakthrough bleeding in early pregnancy, which may be mistaken for a period. Fetal ultrasound is the most precise current measure of GA, but is limited by both timing and access to resources. GA dating is more accurate the earlier an ultrasound is performed, and optimal pregnancy dating can be achieved prior to 20-weeks. However, this also requires both sophisticated equipment and well-trained sonographers. Thus, more affordable, accessible, and accurate GA dating methodology represents an unmet clinical need, particularly for pregnant women across diverse socio-economic backgrounds.

Recent developments in omics profiling technology provide new possibilities for characterization of both normal and high-risk pregnancies. Pregnancy is a highly dynamic programmed process that induces a broad spectrum of changes in the maternal transcriptome, proteome, and metabolome. Notably, the metabolome, as the direct outcome of diverse biochemical reactions, is a highly sensitive readout of metabolic regulation during pregnancy. Investigation of longitudinal maternal metabolomic alternations over the course of pregnancy has the potential to be a highly informative approach for mechanistic investigation and a breakthrough tool for GA dating. This approach has recently attracted more attention but has relied mostly on maternal blood samples. Use of maternal urine for GA dating and metabolic profiling has yet to be explored and may provide a cost effective and non-invasive method that could be easily translated into clinical settings. If found to be useful, it would transform the prenatal care, especially in under-resourced regions.

Thus far, pregnancy-related metabolic research has focused primarily on identifying biomarkers as indicators of risk for adverse pregnancy outcomes like preeclampsia, preterm birth, and gestational diabetes. Emerging data suggests, however, that increased surveillance of metabolomic changes during pregnancy may also provide improved opportunities for understanding of maternal metabolic alterations at differential gestational ages (GA), stratify risk-based biomarkers by clinical benefit, and to better elucidate the pregnancy process.

In this study, longitudinal urine samples were profiled, which were collected from 36 pregnant women receiving prenatal care in public and private clinics in San Francisco and annotated a large number of urine metabolites correlated with pregnancy progression.

### Materials and Methods

Sample collection: 346 urine samples were collected at multiple time points during the pregnancy process (11.8-40.7 weeks) and postpartum period for 36 healthy women. The SMART-D cohort represents an ethnically diverse group of participants with a wide range age and BMI distribution. The samples were collected longitudinally and delivered to analysis into two batches.

MS-grade water, methanol and acetonitrile were purchased from Fisher Scientific (Morris Plains, NJ, USA). MS-grade acetic acid was purchased from Sigma Aldrich (St. Louis, MO, USA). Analytical grade internal standards were purchased from Sigma Aldrich (St. Louis, MO, USA). The internal standard mixture of acetyl-d3-carnitine, phenylalanine-3,3-d2, tiapride, trazodone, reserpine, phytosphingosine, and chlorpromazine was 1:50 diluted with 3:1 acetonitrile and water for HILIC, and water for RPLC.

Urine samples were thawed and centrifuged at 17,000 rcf for 10 min. 250 μL supernatant was diluted with 750 μL internal standard mixture, vortex for 10 seconds and centrifuged at 17,000 rpm for 10 min at 4° C. The supernatant was taken for subsequent LC-MS analysis. A quality control (QC) sample was generated by pooling up all the samples and injected between every 10 sample injections to monitor the consistency of the retention time and the signal intensity.

Hypersil GOLD HPLC column and guard column was purchased from Thermo Scientific (San Jose, CA, USA). Mobile phases for RPLC consisted of 0.06% acetic acid in water (phase A) and MeOH containing 0.06% acetic acid (phase B). Metabolites were at a flow rate of 0.25 mL/min, leading to a backpressure of 120-160 bar at 99% phase A. A linear 1-80% phase B gradient was applied over 9-10 min. The heating temperature of the column was set to 60° C. and the sample injection volume was 5 μL.

MS acquisition was performed on an Q Exactive HF Hybrid Quadrupole-Orbitrap mass spectrometer (Thermo Scientific, San Jose, CA, USA) cooperating in both the positive and negative ESI mode (acquisition from m/z 500 to 2,000) with a resolution set at 30,000 (at m/z 400). The MS2 spectrum of the QC sample was acquired under different fragmentation energy (25 eV and 50 eV) of the top 10 parent ions.

Raw data processing. First, all the MS raw data (.raw format) were converted to .mzXML (MS1 raw data) and .mgf format data (QC MS2 data) using ProteoWizard software (proteowizard.sourceforge.net/). Second, all the .mzXML format data was grouped into 3 folders (named as “Blank”, “QC” and “Subject”) and then subjected for the peak detection and alignment. Third, the peak detection and alignment were performed as follows: Briefly, the peak detection and alignment were performed using the centWave algorithm (R package xcms, version 3.8.1). The key parameters were set as follows: method=“centWave”; ppm=15; snthr=10; peakwidth=c(5, 30); snthresh=10, prefilter=c(3, 500); minifrac=0.5; mzdiff=0.01; binSize=0.025 and bw=5. Finally, the generated MS1 peak table includes the mass-to-charge ratio (m/z), retention time (RT), and peak abundances for all the samples, and other information. This MS1 peak table is used for the next data cleaning.

Data cleaning. The data cleanings of the peak table were also performed. First, the peaks detected in less than 20% QC samples were removed from the peak table as noisy. Second, the samples with more than 50% missing values were removed as outlier samples. Third, the remaining missing values (NA) were imputed using the k-nearest neighbors (KNN) algorithm (R package impute). Then, the peak intensity was divided by the mean peak intensity for data normalization to remove the unwanted analytical variations occurring intra-batches. Finally, the ratio of mean values of each peak in two batches was utilized as the correct factor to do data integration. The data processing and data cleaning, script, and parameter setting can be found in GitHub (github.com/jaspershen/metflow2).

The majority of the statistical analysis and data visualization is performed using Rstudio (version 1.2.5019) and R language (version 3.6.0) in a Windows 10×64 OS. Most of the R packages and their dependencies used in this study are maintained in CRAN (cran.r-project.org/) or Bioconductor (bioconductor.org/). The directory used R packages are plyr (version 1.8.5), stringr (version 1.4.0), dplyr (version 0.8.3), purrr (version 0.3.3), readr (version 1.3.1), readxl (version 1.3.1), tidyr (1.0.0), tibble (version 2.1.3), ggplot2 (version 3.2.1), ggsci (version 2.9), patchwork (version 1.0.0), and igraph (1.2.4.2). The main script for analysis and data visualization in this study is provided in GitHub (github.com/jaspershen/smartD_project).

In general, before all the statistical analysis, the data are first log 10 transformed and then auto scaled as flow:

$${{I}^{′}}_{m} = \frac{{I}_{m} - mean ( {\sum }_{m = 2}^{M} {I}_{m} )}{sd ( {I}_{m = 1 to M} )} \qquad ( 1 )$$

The categorical data are described as the frequency counts and percentages, and the values of all continuous variables are presented as the mean plus or minus the standard deviation (SD) or standard error of the mean (SEM). Most metabolic peaks showed right-skewed distribution; thus, the nonparametric methods (Wilcoxon rank-sum test, spearman correlation) are utilized for non-parametric statistical tests. All the P-values are adjusted utilizing False Discovery Rate (FDR, R base function p.adjust). PCA analysis is performed utilizing the R base function prcomp. The R package ggplot2 (version 3.2.21) was used to perform most of the data visualization in this study.

To find the metabolic peaks which significantly changed according to GA during pregnancy, significance analysis of microarray (SAM) and linear regression model between GA and metabolic peaks were utilized. SAM assigns a score to each metabolic peak on the basis of change in peak expression relative to the standard deviation of repeated measurements. For metabolic peaks with scores greater than an adjustable threshold, SAM uses permutations of the repeated measurements to estimate the percentage of metabolic peaks identified by chance, the false discovery rate (FDR). SAM was performed utilizing the SAM function in R package samr, and resp.type was set as “Quantitative” and FDR was set as 0.05 with 1,000 permutation tests. For the linear regression model, the R base function lm was utilized. To adjust the potential confounders, the participants' baseline, namely acquisition batch, BMI, mother age, parity, and ethnicity were also imputed into the linear regression model, and the metabolic peaks with FDR adjusted P-value<0.05 were selected. Finally, only the 3,020 metabolic peaks (2,436 increased metabolic peaks and 584 decreased metabolic peaks) that are significant in both two methods were used for subsequent K-means consensus-clustering analysis.

Unsupervised K means consensus-clustering of the 302 urine metabolome samples was performed with the R package CancerSubtypes and ConsensusClusterPlus using the 3,020 metabolic peaks that were discovered by SAM and linear regression model. The data was log 10-transformed. Samples clusters were detected based on K-means clustering, Euclidean distance and 1,000 resampling repetitions in ExecuteCC function in the range of 2 to 6 clusters. The generated empirical cumulative distribution function (CDF) plot initially showed optional separation 2 and 3 clusters for all urine samples. And from the consensus matrix heatmaps we can also 2, 3 and 4 clusters seem to have good clustering. To further decide how many clusters (k) should be generated, the silhouette information from clustering was extracted using silhouette_SimilarityMatrix function. k=2, 3, 4 was compared and found that when k=3 high stability for clustering was obtained. So finally, all the urine samples were assigned to 3 clusters.

PIUMet is a network-based tool (fraenkel.mit.edu/PIUMet/) which infers putative metabolites corresponding to features and molecular mechanisms underlying their dysregulation, which means that they can transfer metabolic peak information to network information. For each GA range, the altered metabolic peaks were outputted as txt format files with three columns: m/z, polarity, and −log 10 (FDR adjusted P-value) and then uploaded into the PIUMet website. The parameters are set as below: number of trees: 10, edge reliability: 2, negative prize degree: 0.0005, and number of repeats: 1. Then all the results from PIUMet are processed (github.com/jaspershen/smartD_project). Briefly, all the annotation results from PIUMet for each GA range were combined, and if one metabolite matches more than one metabolic peak, only the metabolic peaks that appeared more than two GA ranges were kept, and other metabolic peaks were removed. Then for each metabolite, the matched metabolic peaks are used to extract quantitative information and the mean values were used quantitative values for this metabolite.

Most metabolic peaks (metabolites) are not normally distributed across all urine samples, and thus the Spearman correlation was used to build the correlation. For the annotation result from PIUMet and dataset combined by metabolite and clinical variables, the correlations between each pair variable were calculated, and then only the absolute correlation>0.5 and FDR adjusted P-value<0.05 were kept to construct correlation networks.

Community analysis was performed using the method based on edge betweenness developed by Girvan and Newman which is embedded in R package igraph. In a network, the edge betweenness score of an edge measures the number of shortest paths through it. So, the idea of edge betweenness based community structure detection is that it is likely that edges connecting separate modules have high edge betweenness as all the shortest paths from one module to another must traverse through them. Briefly, this is an iterative process, in each iteration, the edges with the highest edge betweenness score were removed, and the process was repeated until only individual nodes remain. Finally, a hierarchical map is retrieved: a rooted tree, called a dendrogram of the graph. The leaves of the tree are the individual nodes, and the root of the tree represents the whole graph (network). Then an unbiased method, modularity of the detected community structure was used to analyze the correlation network at a cut level. The modularity of community structure corresponds to an arrangement of edges that is statistically improbable when compared to an equivalent network with edges placed at random. At every iteration of the community analysis, the modularity was computed and the communities were analyzed at the iteration which maximized this quantity. To make sure that the findings are robust and reliable, only the communities (or clusters) with at least 3 modes were kept for subsequent analysis. All the networks were visualized using R package ggraph (version 2.0.0). mean values were used as quantitative values for this metabolite.

The human KEGG pathway database is downloaded from KEGG (www.genome.jp/kegg/) utilized R package KEGGREST. The original KEGG database has 275 metabolic pathways, and then it was separated into metabolic pathways and disease pathways based on the “Class” information for each pathway. The pathways with “Human Disease” class were assigned into the disease pathway database, which contains 74 pathways and remained 201 pathways were assigned into metabolic pathway database. The pathway enrichment analysis is used in the Hypergeometric distribution test. P-values are adjusted by the FDR method and the cutoff was set as 0.05.

To achieve accurate metabolite annotation for this study, three criteria are used for metabolite annotation: (1) accurate mass (m/z), (2) retention time (RT) and (3) MS2 spectral similarities. The public MS2 spectral databases have no retention times for standards, so only the accurate mass and MS2 spectral similarity are used. For each matching, the match score was calculated to represent the match similarity. Each score gives the standardized range from 0 to 1, meaning from no match (0) to a perfect match (1), respectively. Then the remaining unannotated metabolic peaks were matched with the online databases NIST (chemdata.nist.gov/) and METLIN.

For the metabolite annotated, accurate mass (m/z), retention time (RT) and MS2 spectra was obtained, so the annotations are level 1 according to MSI. For metabolites annotated using the public databases, only accurate mass and MS2 spectra are used for matching, so the annotation is level 2 according to MSI.

Data organization. All the MS2 spectra (.mgf format) from QC samples were matched with MS1 peaks in peak table according to accurate mass (m/z, tolerance is set as ±25 ppm) and RT (tolerances is set as ±10 seconds) using the code provided by MetDNA. If one MS1 peak matches multiple MS2 spectra, only the most abundant MS2 spectrum is kept. Finally, the generated MS1/MS2 pairs were used to match with private and public MS2 spectral databases (HMDB [www.hmdb.ca/], MoNA [mona.fiehnlab.ucdavis.edu/], and MassBank [massbank.eu/MassBank/]).

Accurate mass and RT match score. The match tolerance for the MS1 m/z value is set as ±25 ppm and RT match tolerance is set as ±10 seconds. Only the metabolites that meet those tolerances are kept. The match scores refer to MS-DIAL and are calculated as follows:

$$Accurate mass ( m / z ) or RT match score = exp [ - 0.5 ( \frac{experimental value - standard value}{δ} ) ] \qquad ( 2 )$$

Where the experiment value is the experimental m/z or RT from MS¹ peak table, and the standard value is the standard m/z or RT from MS² spectral databases. These equations assume that for accurate mass and retention time match scores, the differences between experimental and standard values follow the Gaussian distribution (normal distribution). The standard deviation δ is the accurate mass (m/z) or RT match tolerance.

MS2 spectral match score. The MS2 spectral match score is a combined value of three scores, namely forward dot-product (DPf), reverse dot-product (DPr), and the matched fragments ratio (MFR). Both the DP scores and MFR ranges are from 0 to 1, meaning from no match (0) to a perfect match (1). The intensities of the fragment ions in the MS2 spectra are rescaled so that the highest fragment ion is set from 0 to 1, meaning from no match (0) to a perfect match (1). The intensities of the fragment ions in the MS2 spectra are rescaled so that the highest fragment ion is set to 1.

The forward and reverse dot-product are calculated as follow:

$$Dot product ( DP ) = \frac{\sum {W}_{S} {W}_{E}}{\sqrt{\sum {W}_{S}^{2} {W}_{E}^{2}}} \qquad ( 3 )$$

Where the weighted intensity vector, W=[relative intensity of fragment ion]n[m/zvalue]m, n=1, m=0; S=standard and E=experiment. DP from both forward and reverse matches are generated using this equation.

The matched fragment ratio (MRF) is utilized to assess how many fragments are matched in all fragments in both experiment and standard MS2 spectra and is calculated as follow:

$$Matched fragment ratio = \frac{{W}_{S} ⋂ {W}_{E}}{{W}_{S} ⋃ {W}_{E}} \qquad ( 4 )$$

Where the weighted intensity vectors are the same as the equation (2). W_{S}∩W_{E} mean the number of matched fragments between standard and experiment MS2 spectra, and W_{S}∩W_{E} mean the number of all the fragments in standard and experiment MS2 spectra.

Finally, the MS2 spectral match score is combined the forward DP (DP_{f}), reverse DP (DPᵣ) and matched fragment ratio (MFR), and the weight for forward DP (W_{f}), reverse DP (Wᵣ), and matched fragments ratio (Wₘ) are set as 0.3, 0.6 and 0.1, respectively.

MS² spectral match score=W_{f}×DP_{f}+Wᵣ×DPᵣ+Wₘ×MFR (5)

Total match score. Three match scores, namely accurate mass, retention time, and MS2 spectra match scores, are used to calculate the total match score as follow:

Total match score=W_{m/z}×S_{m/z}+W_{RT}×S_{RT}+W_{MS2}×S_{MS2} (6)

Where W_{m/z}, W_{RT}, and W_{MS2} are weighted for accurate mass (S_{m/z}), RT (S_{RT}), and MS2 (S_{MS2}) spectral match scores, and set as 0.25, 0.25, and 0.5, respectively. For public MS² spectral databases without RT information, the above three weights are set as 0.375, 0, and 0.625, respectively.

If one metabolic peak matches multiple metabolites, the annotated metabolites are sorted according to the total match score. And all the potential metabolite markers were finally checked to confirm the accuracy of metabolite annotation.

To make sure that the selected metabolite biomarkers have the right annotations and have good reproducibility, all the metabolite biomarkers were manually checked. Two criteria, (1) peak shape, and (2) MS2 spectra match are included. Only the metabolite biomarkers with good peak shapes remain for prediction model construction. Metabolite biomarkers that have bad peak shapes may have bad reproducibility, so they are discarded. The metabolite biomarkers that have bad MS2 spectra match with standards are also removed to avoid the wrong annotation.

Feature selection. The Boruta algorithm (R package Boruta, version 6.0.0) is utilized to select potential biomarkers. Briefly, it duplicates the dataset and shuffles the values in each column. These values are called shadow features. Then, it trains a Random Forest classifier on the dataset, and checks for each of the real features if they have higher importance. If it does, the algorithm will record the feature as important. This process is repeated 100 iterations. In essence, the algorithm is trying to validate the importance of the feature by comparing it with randomly shuffled copies, which increases the robustness. This is performed by comparing the number of times a feature did better with the shadow features using a binomial distribution. Finally, the confirmed features are selected as potential biomarkers for Random Forest model construction.

Parameter optimization. All the parameters are used as default settings except ntree (i.e., number of trees to grow) and mtry (i.e., number of variables randomly sampled as candidates at each split) in the Random Forest model (R package randomForest, version 4.6-14). Those two parameters are optimized on the training dataset. The two parameters are combined together to form a set of parameter combinations. The performance of each parameter combination is evaluated using the mean squared error (MSE). The parameter combination with the smallest MSE is used to build the final prediction model.

Gestation age (GA) prediction model. All the samples acquired in batch 1 (16 subjects and 125 samples) are used as the training dataset. All the samples acquired in batch 2 (20 subjects and 156 samples) are used as the validation samples. First, the training dataset is utilized to get the potential biomarkers using the feature selection method described above. Then a Random Forest prediction model is built based on the training dataset. Then based on this prediction model, a linear regression model between predicted GA and actual GA was also constructed. Then the predicted GA from Random Forest is corrected by this linear regression model. So, the GA prediction model contains two models, namely Random Forest and linear regression model. Then the external validation model is utilized to demonstrate its prediction accuracy. The predicted GA and actual GA for the validation dataset are plotted to observe the prediction accuracy. Then the RMSE (root mean squared error) and adjusted R2 are used to quantify the prediction accuracy.

For internal validation, the bootstrap sampling method is utilized. Briefly, the same number of samples from the training dataset were randomly sampled with replacement (about 63% of the unique samples on average) and then used as an internal training dataset to build the Random Forest prediction model using the same selected features and optimized parameters. The remaining about 37% of the samples on average were used as internal validation data. Those steps repeat 1,000 times. Finally, for each sample, we got more than one predicted GA value. The mean value of multiple predicted GA values is used as the final average predicted GA and used to calculate MSE and adjusted R2.

Sampling time to the delivery prediction model. Sampling time to delivery (week) is defined as the time difference between the delivery date and sample collection date. So, for each sample, time to delivery is calculated, which was used as responses to build a prediction model. All the steps are the same as the GA prediction model.

The permutation test was utilized to calculate p-values to judge whether the random forest prediction models that were constructed are overfitting. First, all the responses (GA or time to delivery in this study) are randomly shuffled for both training and validation datasets, respectively. Then the potential biomarkers are selected and the parameters of random forest are optimized in the training dataset using the method described above. Thirdly, the random forest prediction model is built using the selected features and optimized parameters in the training dataset. Finally, this random forest prediction model was used to get the predicted responses for the validation dataset. Then the null RMSE and adjusted R2 was obtained. This process was repeated 1,000 times, resulting in 1,000 null RMSE and 1,000 null adjusted R2 vectors. Using maximum likelihood estimation, these null RMSE values and adjusted R2 values are modeled as Gamma distribution, and then the cumulative distribution function (CDF) is calculated. Finally, the P-values for the real RMSE and adjusted R2 are calculated from the null distributions, respectively.

The fuzzy c-means clustering algorithm (R packages e0171 and Mfuzz) is utilized to cluster the metabolite biomarkers into different classes and explore the metabolite changes according to the gestation age (weeks). Because the participants' samples were collected at different time points, so all the samples are grouped to different time ranges. The time ranges are from 11 weeks to 41 weeks and step is two, and the postpartum samples are grouped to the “PP” group. For the samples in the same time range group, each metabolite's intensity is calculated by the mean value of all the samples in this group. So finally, a new data frame with 16 new observations was obtained. First, the parameter “m” (the degree of fuzzification) was optimized based on a method using the Mfuzz package. The optimal cluster number is determined based on the within-cluster sum of squared error. Then all default parameters were used to build the fuzzy c-means clustering. For each cluster, only the features with a membership score>0.5 were considered; this high stringency was chosen so that the dynamics of the core members of each cluster can be explored. In fuzzy c-means clustering, the membership score is the probability of a feature belonging to any cluster, each feature is assigned a cluster based on its top membership score (as opposed to k-means clustering, where the membership score is binary). The color of each feature is directly based on the membership score (from blue to red, membership score from low to high). The output results were not smoothed.

### Results

### Study Design

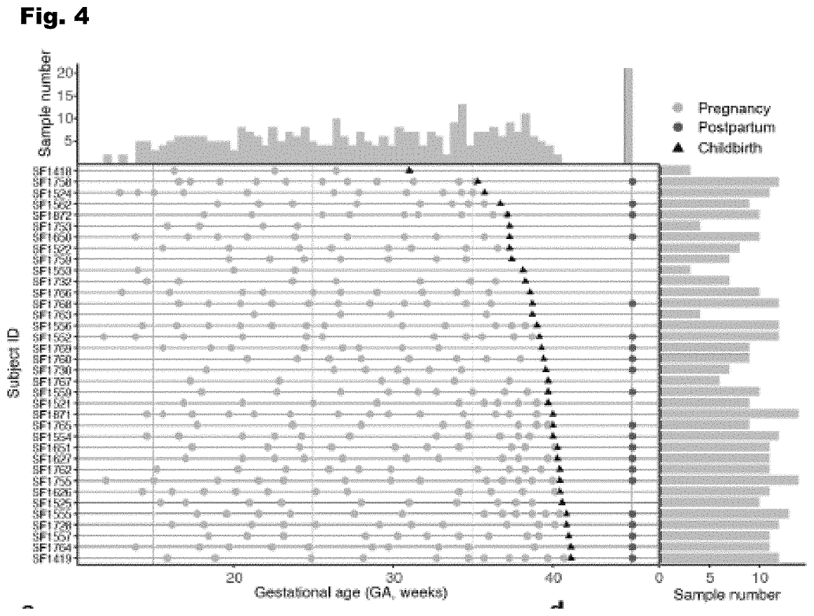

This observational study aimed to assess whether the urine metabolome in pregnancy could be used to identify dynamic metabolic changes during pregnancy and predict GA by week. To do this, urine samples were collected from 36 pregnant women receiving prenatal care in San Francisco who were recruited into the SMART Diaphragm (SMART-D) study between November 2014 and October 2018 (FIG. 4). The SMART-D study developed and iterated a patient-controlled, vaginally inserted device that detects microscopic changes in cervical collagen structure to provide earlier predictions of preterm birth risk and open a new potential treatment window. Diverse samples were collected during longitudinal visits over the course of pregnancy and postpartum periods, including urine samples, cervical-vaginal swabs, etc., together with detailed clinical and device measurement data. Urine samples used for analyses in the present study were collected as part of the SMART-D study protocol wherein at least one urine sample from each participant was collected for each trimester. Each participant contributed 3-13 samples throughout pregnancy; overall, each week of pregnancy after 15 weeks was represented by at least one sample across participants (FIG. 4). High-resolution liquid chromatography-mass spectrometry (LC-MS) was used to characterize the metabolome of all collected urine samples.

Participants of diverse backgrounds were included in the SMART-D study from which these samples are derived. The 36 participants in the cohort were comprised of five ethnicities (Asian, Black, Latina, Pacific Islander, and White), with ages ranging from 21 to 39 years old. The pre-pregnancy body mass index (BMI) of participants varied from 19.5 to 57.2. Parity ranged from 1 to 9. The high-density sampling design of the SMART-D study enabled the close monitoring of dynamic metabolome alterations of women throughout pregnancy.

### The Urine Metabolome Accurately Reflects Metabolic Alterations During Pregnancy

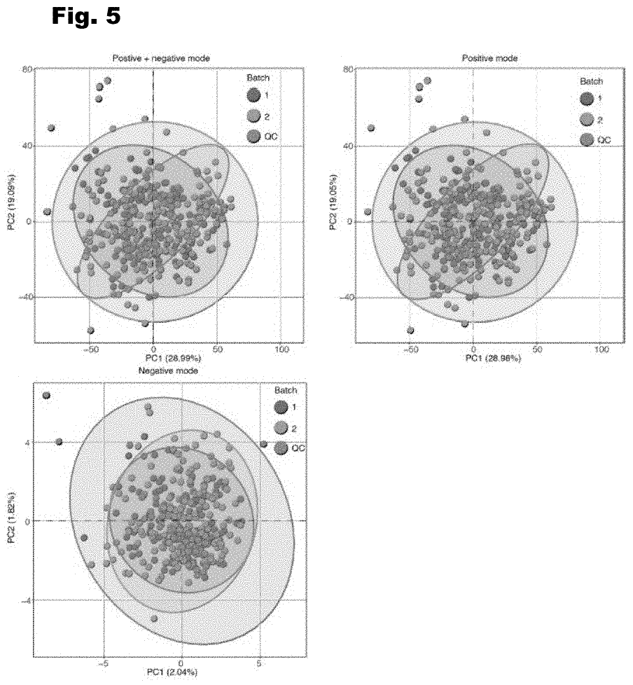

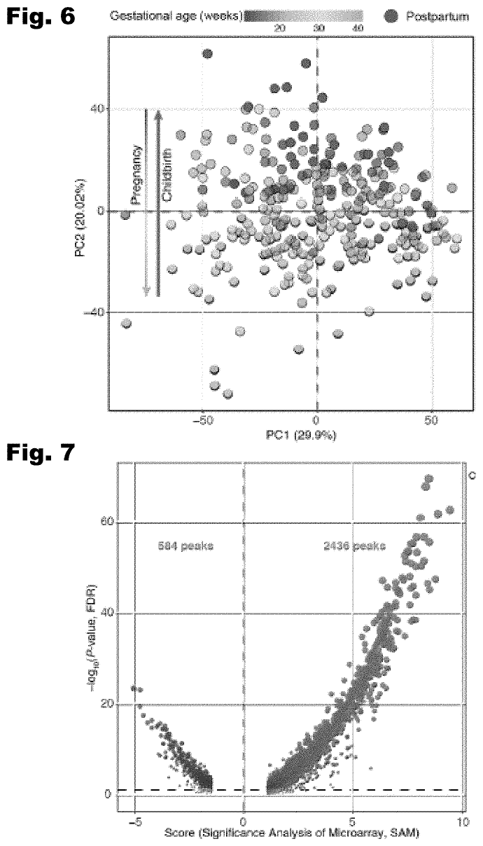

Untargeted high-resolution metabolomics was performed on all collected urine samples. After data processing (peak detection and alignment) and cleaning (missing value processing, normalization and batch integration, outlier removal), 20,314 metabolic peaks (or metabolic features, characterized by unique accurate mass and retention time) were detected including 15,398 and 4,916 metabolic peaks in positive and negative modes, respectively. Forty-four samples were removed as outliers; 302 samples remained for all subsequent analyses. Quality of urine metabolomics data was assessed using Principal Component Analysis (PCA), which showed no batch effect. Additionally, most QC samples clustered tightly in the center among samples in positive, negative, and combined datasets (FIG. 5), indicating the high quality of our acquired metabolomics dataset. In addition, PCA including all metabolic peaks with QC RSD<30% revealed a continuous separation between samples from early and later GA (FIG. 6). Interestingly, the postpartum urine samples most closely resemble early GA urine samples (FIG. 6). Additionally, most individual participants followed the same patterns of metabolic change as the overall dataset.

Overall metabolome alterations during pregnancy were then examined. Significance analysis for microarrays (SAM) and linear regression model (acquisition batch, BMI, mother age, parity, and ethnicity were adjusted as confounders) were utilized to discover altered metabolic peaks during pregnancy (SAM FDR<0.05 and linear regression model FDR<0.05, Methods). 14.87% of all detected metabolic peaks (3,020 out of 20,314 peaks, with 2,436 and 584 metabolic peaks in positive and negative modes, respectively) were significantly altered during pregnancy (FIG. 7). Altered metabolic peaks were then used for unsupervised k-means consensus-clustering (FIG. S4, Method). Three robust clusters clearly correlated with gestational age were detected, namely cluster 1: 10-26 weeks, cluster 2: 26-32 weeks, and cluster 3: 32-42 weeks. Additionally, consistent with results from PCA, almost all samples after childbirth were included in cluster 1, which contains most of the early GA samples, suggesting that the urine metabolome rapidly returned to baseline and reflected early GA patterning. Taken together, these results demonstrated that a high-quality urine metabolome accurately reflects systemic metabolic alterations throughout pregnancy.

### Gestational Age Prediction Using the Urine Metabolome Model

An accurate and noninvasive method of estimating gestational age has the potential to inform prenatal and neonatal care in instances where dating is uncertain. To this end, it was examined whether the urine metabolome could be used to estimate gestational age. Urine samples during pregnancy were assigned to training (16 subjects, 125 samples) validation (20 subjects, 156 samples) datasets by acquisition. The demographics and birth characteristics of training and validation datasets were not significantly different (P-value>0.05).

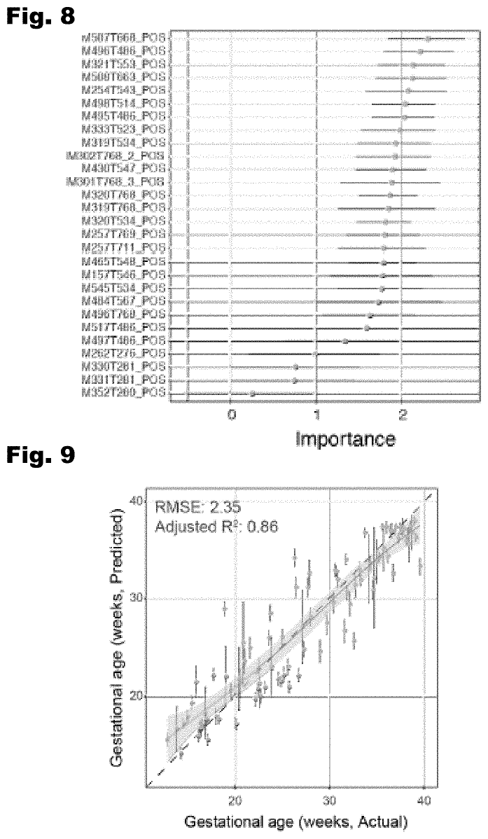

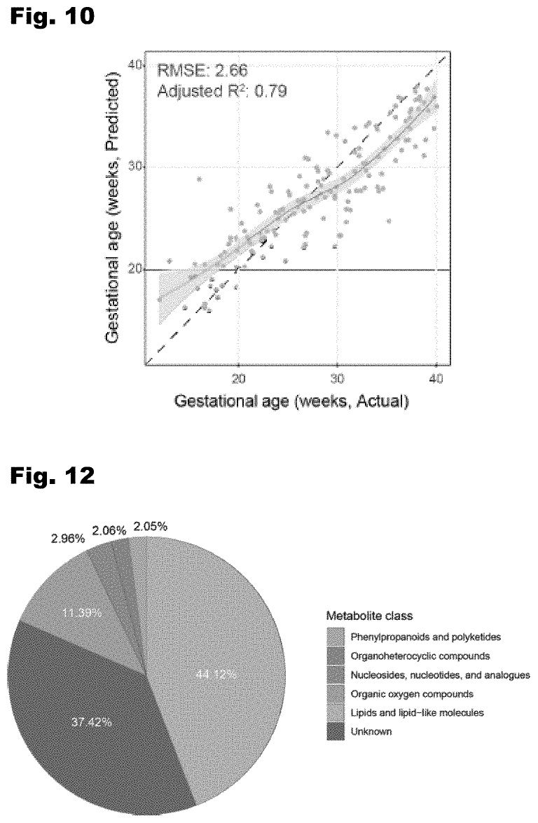

The prediction model was constructed by starting with all metabolic peaks for feature selection based on the Boruta algorithm. The metabolic peaks without acceptable peak shapes were removed, with the remaining 28 metabolic peaks as potential biomarkers. These biomarkers were used to build a Random Forest prediction model (FIG. 8). The training dataset was utilized as the internal dataset and to validate prediction accuracy using the bootstrap method (1,000 times). The root mean squared error (RMSE) between actual and predicted gestational age (weeks) was found to be 2.35 weeks and adjusted R2 was 0.86 (Pearson correlation r=0.93; P-value<2.2×10−6) (FIG. 9). For the external validation dataset, the RMSE was 2.66 weeks and the adjusted R2 was 0.79 (Pearson correlation r=0.89; P-value<2.2×10−6) (FIG. 10). This result demonstrated that the prediction model is not overfitting. Overall, our results demonstrated that the urine metabolome may be useful for accurately predicting gestational age.

The impact of patient demographics on prediction accuracy was also assessed. Maternal BMI, age, parity, and ethnicity were included with 28 metabolic peaks to construct a prediction model. The RMSE of this model was 2.70 and adjusted R2 was 0.76, which demonstrated no significant differences compared to the prediction model utilizing 28 metabolic peaks. Inclusion of subject demographics minimally improved prediction accuracy.

### Prediction of Gestational Age at the Individual Level

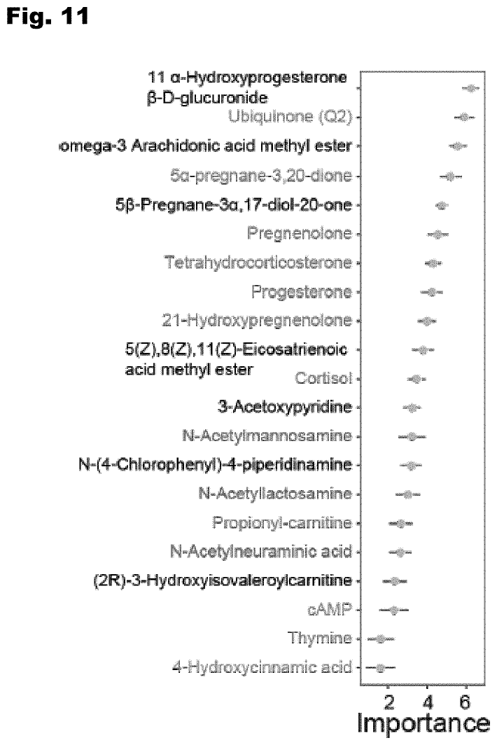

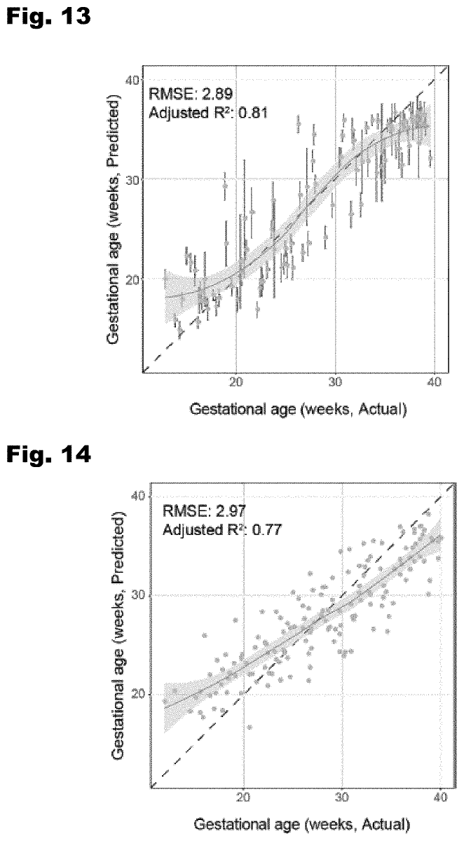

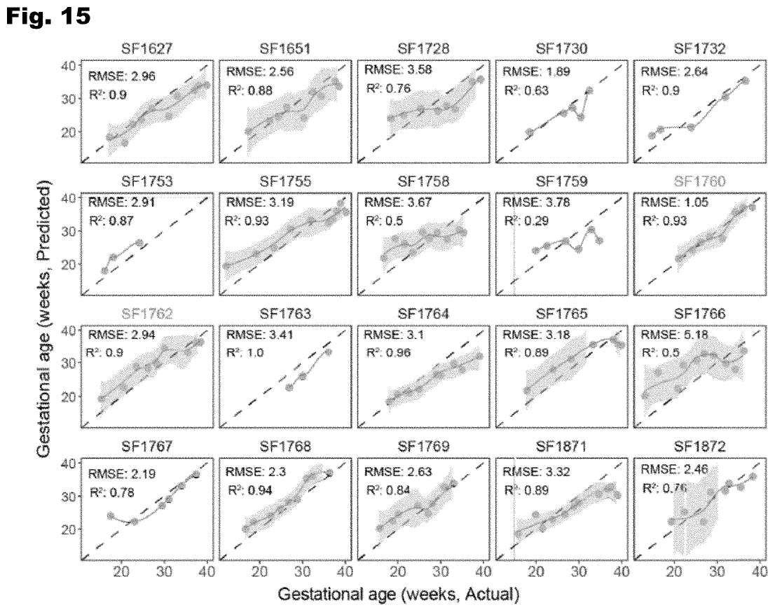

It was demonstrated that the pregnancy urine metabolome could accurately predict gestational age with 28 metabolic peaks using a Random Forest model. Metabolic peaks were annotated using the in-house MS2 pipeline based on in-house and public MS2 databases. While 875 of 20,314 total level 1 or level 2 metabolites were annotated in the full dataset, only 5 out of the 28 metabolic peaks in the final model were annotated. Therefore, the 875 annotated metabolites were utilized to predict gestational age in individual patients. After feature selection based on the Boruta algorithm (see Methods), 32 metabolites were selected as potential biomarkers. To ensure the robustness and reproducibility of the prediction model, metabolites without acceptable peak shapes or without good MS2 spectral matches were excluded. Finally, 21 metabolites were included as the final biomarkers to build the prediction model in the training dataset (FIG. 11). To create an overview of the 21 metabolite biomarkers, the Classyfire algorithm (Y. Djoumbou Feunang, et al., J Cheminform. 2016; 8:61, the disclosure of which is incorporated herein by reference) was utilized to access their chemical class information. Interestingly, most of the metabolite biomarkers were lipids and lipid-like molecules (e.g., hormones), such as 5α-preganane-3, 20-dione, pregnenolone, and progesterone, which is consistent with previous findings from maternal plasma. Most of these metabolites had high ranks in the prediction model (ranks: 2, 4, 6, 7, 8, 9, 11, and 16; importance ratio: 44.12%; FIGS. 11 and 12). Importantly, the 21 metabolite biomarkers achieved a prediction accuracy for gestational age comparable to the model that used metabolic features. Specifically, the adjusted R2 are 0.81 (Pearson correlation r=0.90, P-value<2.2×10−6) and 0.77 (Pearson correlation r=0.87, P-value<2.2×10−6) for internal and external validation datasets, respectively (FIGS. 13 and 14). The RMSE were 2.89 and 2.97 weeks for internal and external validation datasets, respectively. To avoid overfitting, a 1,000-time permutation test was performed, and the results suggest that the model is not overfitting. Intriguingly, it was also found that model performance improved significantly as the pregnancy progressed. As FIGS. 13 and 14 show, the RMSE for both training (RMSE=4.71 for the first trimester, 2.81 for the second trimester, 2.82 for the third trimester) and validation datasets (RMSE=7.30 weeks for T1, 3.14 for T2, 2.81 for T3) increased from the first trimester to the third. In addition, it was found that there was no significant difference in the prediction accuracy compared to the prediction model using metabolic peaks, especially in the validation dataset (metabolite model vs. metabolic peak model: RMSE=2.97 vs. 2.66 weeks and adjusted R2=0.77 vs. 0.79). These results suggest that urine metabolite biomarkers can be used to predict gestational age, which has important potential clinical applications. These findings were also applied to gestational age estimation for individual participants. For the external validation dataset, 16 of the 20 participants had adjusted R2s larger than 0.75 (FIG. 15). These results indicate that the prediction model is also robust for individual prediction. Since the cohort includes women with diverse demographic and clinical characteristics, this also suggests that the prediction model has utility for pregnant women from diverse backgrounds. The cohort includes a nearly two-decade age range, infant birth weight from 1,940.0 to 6,185.0 grams (IQR: 511.25), pre-pregnancy BMI from 19.49 to 57.23 (IQR: 8.39), and parity from 1 to 9 (IQR: 2). The impact of these personal characteristics was evaluated on prediction accuracy at an individual level. The correlations between RMSE/adjusted R2 and continuous characteristics were calculated. Surprisingly, the continuous characteristics, namely, age (maternal age at birth), birth weight, pre-pregnancy BMI, and parity are not significantly correlated with prediction accuracy (Pearson correlation, all absolute correlations<0.5 and all P-values>0.05). Importantly, it was found that there are three outlier participants for birth weight, BMI, and parity, respectively. For participant S1760, the BMI is 57.23 (mean of all: 27.09), which is significantly different from that of most of the participants (P-value<0.001). Notably, the prediction model still achieved high prediction accuracy for this participant (RMSE=1.05, adjusted R2=0.93; FIG. 15). For participant S1762, who had a parity of nine (mean of all: 2.92, P-value<0.001), good prediction accuracy was also achieved (RMSE=2.94, adjusted R2=0.90; FIG. 15). It was also tested whether categorical characteristics, such as ethnicity, affected prediction accuracy. The results show that the prediction accuracy appeared to be unaffected by those characteristics. (ANOVA test, all P-values>0.05). Taken together, these findings demonstrate that the prediction model for gestational age based on metabolite biomarkers is very robust and can accommodate diversity at an individual level.

### Prediction of Time to Delivery

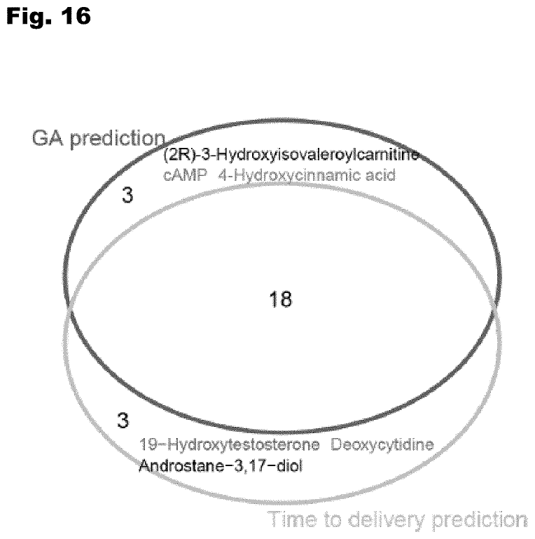

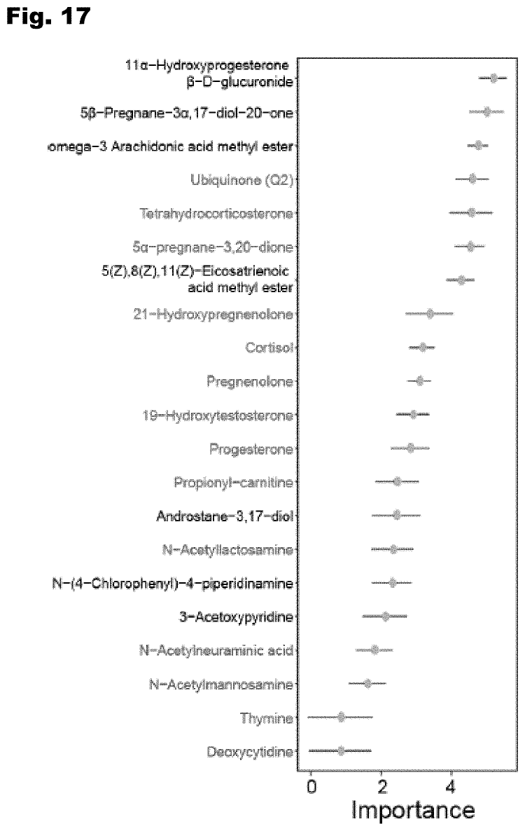

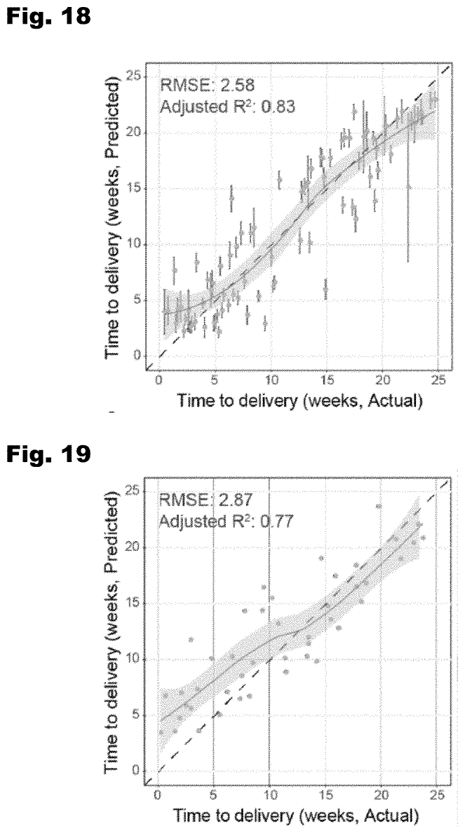

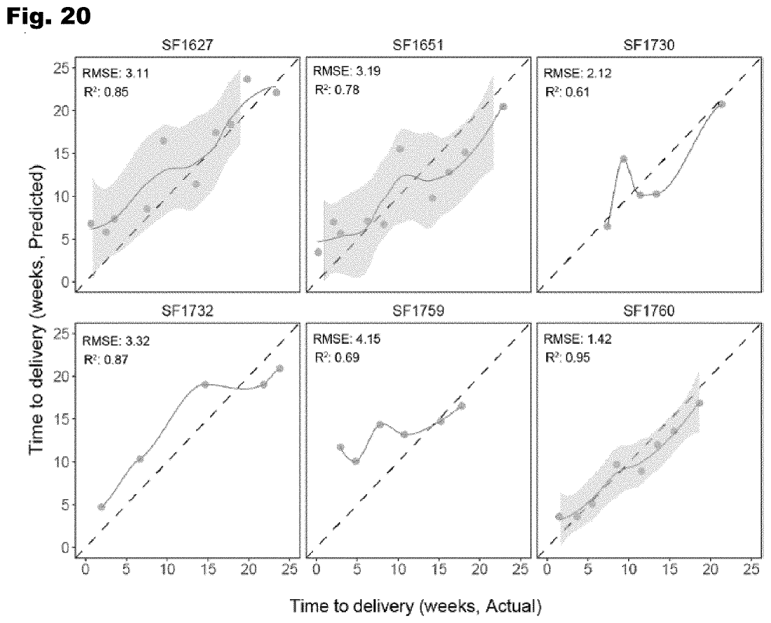

It was next tested whether the urine metabolome could predict time-to-delivery using annotated metabolites. “Time-to-delivery” was defined as the difference between the gestational age at sample collection and gestational age at delivery, which is a criterion independent of ultrasound-estimated gestational age. In this test, the participants who had scheduled Cesarean sections were removed from the dataset and then the remaining 20 participants (14 subjects for training and 6 for validation datasets, respectively) were used for prediction model construction and. Finally, 21 metabolites were included, 18 of which overlapped with the metabolite markers in the prediction model for gestational age (FIGS. 16 and 17). The values predicted by the model agreed with actual values for both training (RMSE=2.58 weeks; adjusted R2=0.83; Pearson correlation r=0.94, P-value<2.2×10−6) (FIG. 18) and validation dataset (RMSE=2.87; adjusted R2=0.77; Pearson correlation r=0.88, P-value=4.91×10−15) (FIG. 19), showing accurate time-to-delivery prediction. The permutation test also shows that this model does not overfit the data. Interestingly, the prediction accuracy was independent of study patient demographics, much like the case of the prediction model for gestational age (FIG. 20). These results demonstrate that the prediction model for sampling time-to-delivery based on metabolite biomarkers is also very robust and accounts for diverse characteristics on an individual level.

### Altered Metabolic Signatures During Pregnancy

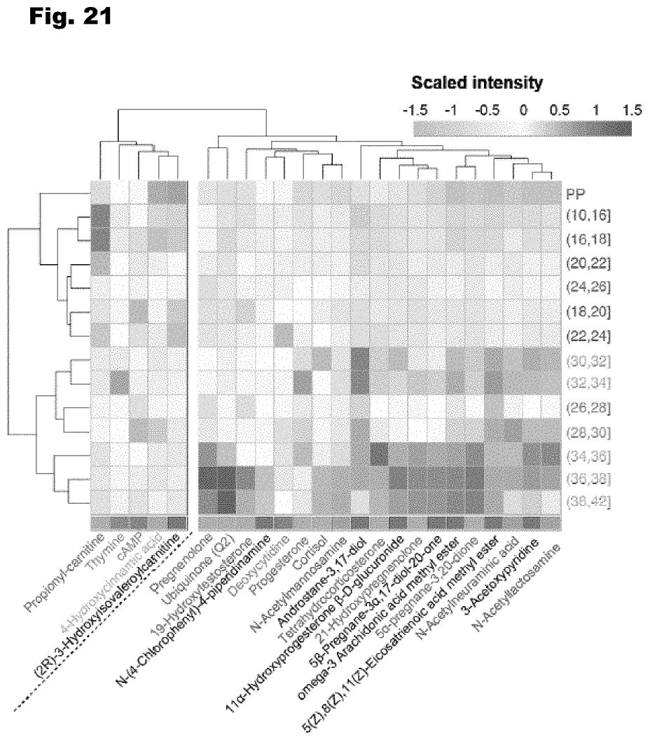

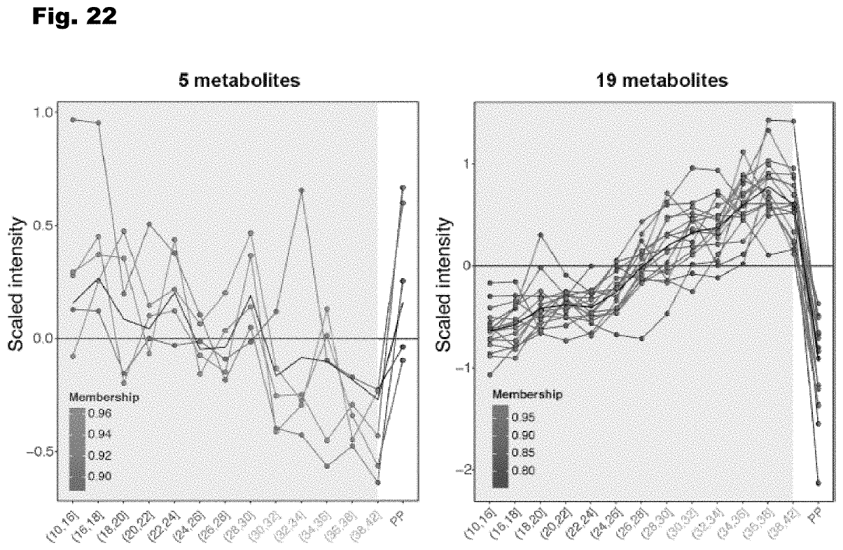

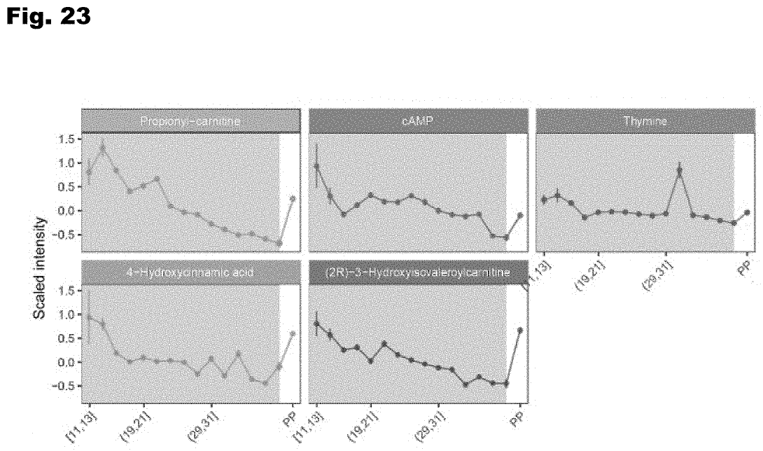

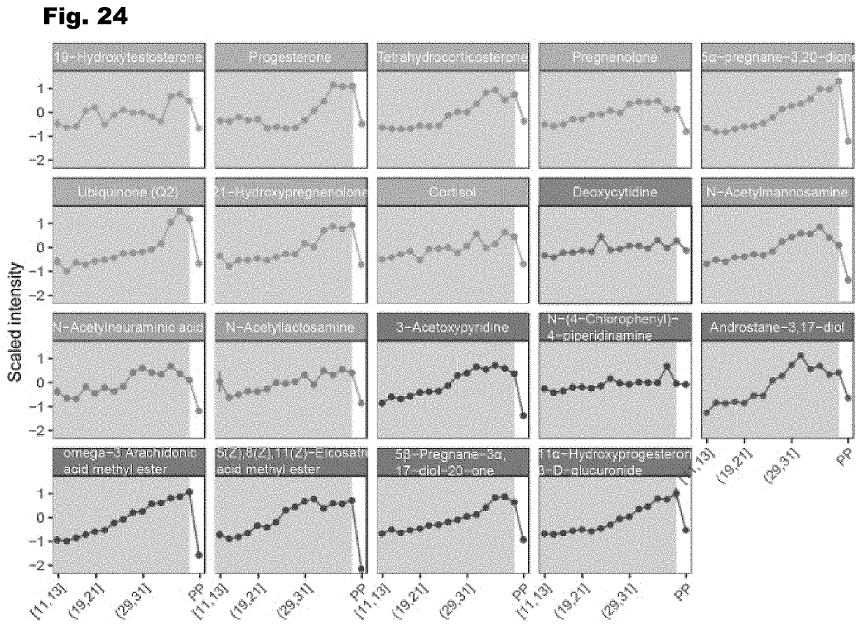

The biological function of the 24 metabolite markers that were found to differ significantly as pregnancy progressed were further explored. First, the Classyfire algorithm was utilized to determine the chemical class information of all 24 metabolite biomarkers. Most of the metabolite biomarkers (9 out of 24, 37.5%, 8 are unknown) are lipids and lipid-like molecules (hormone), which is consistent with our finding above at the metabolic feature level. To capture the altered metabolic signatures during pregnancy, the hierarchical clustering and fuzzy c-mean clustering algorithms were utilized to group the 24 metabolite markers, which clustered into two groups with contrasting regulation patterns during pregnancy progression (FIGS. 21 and 22). The first group was downregulated during pregnancy but increased to normal levels postpartum, including a panel of carnitines and signaling compounds such as cAMP (FIG. 23) whereas the second group demonstrated increased abundance as the pregnancy progressed and then fell to normal levels postpartum (FIG. 24). This group comprises diverse hormones and intermediates, such as 19-hydroxytestosterone, cortisol, pregnenolone, 5α-pregnane-3,20-dione, etc. These hormones were highly enriched in the glucocorticoid and mineralocorticoid biosynthesis, growth hormones, and lipid metabolism and signaling pathways. Some metabolic markers, progesterone for instance, have been applied in clinical tests for therapeutic treatment of preterm birth and pregnancy loss. These data suggest that other members of the steroid group with similar regulation behavior as progesterone could also serve as potential candidates for diagnostic monitoring or therapeutic targeting during pregnancy. Correlation analysis of the metabolome at different GA periods showed overarching significant alterations as the pregnancy progressed. The early stages of pregnancy showed a positive correlation between the metabolite intensity and GA, while the late stage showed a negative correlation. Their relative distances to postpartum levels also showed a similar pattern. This suggested significant alteration of urine metabolome is common in the later stages of pregnancy with the potential to precisely predict delivery time. The correlation between different metabolic markers demonstrated positive correlations between most markers and some potential delivery-related factors including maternal BMI and birth weight, indicating co-regulation of most metabolic pathways, except for the negative correlation between BMI and pregnenolone. Several studies have suggested a higher risk of preterm birth among obese women. Thus, the examination of pregnenolone levels could aid in GA prediction and preterm birth risk. BMI is negatively correlated with most lipid metabolite biomarkers, although only BMI-Pregneolone demonstrated significant correlation here (FDR adjusted P-value<0.05, FDR). In fact, BMI was shown to exhibit negative correlations with other lipids, except 19-Hydroxytestosterone, but none of these trends were statistically significant (FDR adjusted P-value>0.05).

### CONCLUSIONS

A reliable estimate of gestational age is critical for the provision of preventive prenatal health care and appropriate interventions as the medical needs of the mother and fetus change throughout pregnancy. Although substantial work has been done to elucidate the dynamic metabolic pathways of pregnancy progression using collected blood samples, the dynamic pregnancy urine metabolome has been only sparsely characterized. For this study, an unbiased comprehensive metabolic profiling approach was applied to analyze urine samples from pregnant women who were participants in the SMART-D cohort to better understand prenatal and post-natal metabolic dynamics in maternal urine. Models to estimate gestational age at the time of sampling and predictions for time-to-delivery from sampling were developed. Metabolic models for gestational age at time-to-delivery were validated at the cohort and individual levels and found to be highly predictive (training database: adjusted R2 0.81, RMSE 2.89; test database: R2 2.97, RMSE 0.77) (FIGS. 14 and 15). Importantly, prediction was found to improve with increased sampling frequency. The gestational age model tended to overestimate gestational age during early pregnancy and underestimate gestational age in later pregnancy. These discrepancies may be due to underlying heterogeneous biological processes happening throughout pregnancy and will need further investigation in a larger population. Overall, the findings suggest that the pregnancy urine metabolome can be successfully leveraged to estimate gestational age at sampling and to predict time-to-delivery.

Pregnenolone, progesterone and corticoid were all upregulated in the glucocorticoid pathways during pregnancy and related metabolites, used in the time-to-delivery prediction model, were enriched for glucocorticoid and CMP-N-acetylneuraminate biosynthesis pathways. These hormones have been reported to play key roles in pregnancy regulation. For instance, progesterone has been approved for the treatment of amenorrhea, metrorrhagia, and infertility. Furthermore, N-acetylmannosamine and N-acetylneuroaminate were both significantly upregulated in the CMP-N-acetylneuraminate biosynthesis pathway, although the impact of these signaling molecules on pregnancy-related processes remains to be explored.

As a proof-of-principle, the results show that urine metabolomic profiles can be used to track gestation throughout pregnancy. By applying a random forest model, one can successfully predict gestational age based on a panel of urine metabolites, including diverse glucocorticoids, lipids, gluconoids, and amino acid derivatives, indicating comprehensive regulation of glucocorticoid biosynthesis and CMP-N-acetylneuraminate biosynthesis by pregnancy.

Collectively, the characterized alterations in the maternal urine metabolome demonstrated a strong correlation between GA and pregnancy progression. The ability to determine GA accurately and conveniently and to predict time-to-delivery will aid in monitoring fetal development and targeting interventions to improve maternal and infant health outcomes. This study also revealed substantial differences between maternal urine metabolites in healthy pregnancies and those ending in preterm birth, which suggests variation of metabolic regulation mechanisms among different pregnancy outcomes. Close monitoring of maternal metabolism alterations and their correlation with GA has the potential to provide more insight into the programmed regulation of fetal development and the development of pregnancy disorders. The non-invasive nature and accessibility of the urine metabolome enables improved determination of GA without limitation across various clinical settings, including in middle-to-low-income countries where women may have limited access to early prenatal care.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

1. A method for determining gestational age or time-to-delivery of an individual, the method comprising:
obtaining measurements of one or more analytes derived from one or more urine sample collected from an individual to be assessed;
predicting, using a predictive computational model and the measurements of the one or more analytes, a gestational age or a time to delivery of the individual.

2. The method of claim 1, wherein the predictive computational model incorporates one or more of: ridge regression, K-nearest neighbors, LASSO regression, elastic net, least angle regression (LAR), random forest, or principal components analysis.

3. The method of claim 1, wherein the predictive computational model is to predict gestational age and the predictive computational model includes at least one feature that is a measurement of one of the following metabolites: 11α-hydroxyprogesterone β-D-glucuronide; ubiquinone (Q2); omega-3 arachidonic acid methyl ester; 5α-pregnane-3,20-dione; 5 β-pregnane-3α, 17-diol-20-one; pregnenolone; tetrahydrocorticosterone; progesterone; 21-hydroxypregnenolone; 5(Z),8(Z),11(Z)-eicosatrienoic acid methyl ester; cortisol; 3-acetoxypyridine; N-acetylmannosamine; N-(4-chlorophenyl)-4-piperidinamine; N-acetyllactosamine; propionyl-carnitine; N-acetylneuraminic acid; (2R)-3-hydroxyisovaleroylcarnitine; cAMP; thymine; and 4-hydroxycinnamic acid.

4. The method of claim 1, wherein the predictive computational model is to predict gestational age and the predictive computational model includes at least two features that are each independently a measurement of one of the following metabolites: 11α-hydroxyprogesterone β-D-glucuronide; ubiquinone (Q2); omega-3 arachidonic acid methyl ester; 5α-pregnane-3,20-dione; 5 β-pregnane-3α, 17-diol-20-one; pregnenolone; tetrahydrocorticosterone; progesterone; 21-hydroxypregnenolone; 5(Z),8(Z),11(Z)-eicosatrienoic acid methyl ester; cortisol; 3-acetoxypyridine; N-acetylmannosamine; N-(4-chlorophenyl)-4-piperidinamine; N-acetyllactosamine; propionyl-carnitine; N-acetylneuraminic acid; (2R)-3-hydroxyisovaleroylcarnitine; cAMP; thymine; and 4-hydroxycinnamic acid.

5. The method of claim 1, wherein the predictive computational model is to predict time to delivery and the predictive computational model includes at least twenty-one features that are each independently a measurement of one of the following metabolites: 11α-hydroxyprogesterone β-D-glucuronide; ubiquinone (Q2); omega-3 arachidonic acid methyl ester; 5α-pregnane-3,20-dione; 5 β-pregnane-3α, 17-diol-20-one; pregnenolone; tetrahydrocorticosterone; progesterone; 21-hydroxypregnenolone; 5(Z),8(Z),11(Z)-eicosatrienoic acid methyl ester; cortisol; 3-acetoxypyridine; N-acetylmannosamine; N-(4-chlorophenyl)-4-piperidinamine; N-acetyllactosamine; propionyl-carnitine; N-acetylneuraminic acid; (2R)-3-hydroxyisovaleroylcarnitine; cAMP; thymine; and 4-hydroxycinnamic acid.

6. The method of claim 1, wherein the predictive computational model is to predict time to delivery and the predictive computational model includes at least one feature that is a measurement of one of the following metabolites: 11α-hydroxyprogesterone β-D-glucuronide; 5 β-pregnane-3α, 17-diol-20-one; omega-3 arachidonic acid methyl ester; ubiquinone (Q2); tetrahydrocorticosterone; 5α-pregnane-3,20-dione; 5(Z),8(Z),11(Z)-eicosatrienoic acid methyl ester; 21-hydroxypregnenolone; cortisol; pregnenolone; 19-hydroxytestosterone; progesterone; propionyl-carnitine; androstane-3,17-diol; N-acetyllactosamine; N-(4-chlorophenyl)-4-piperidinamine; 3-acetoxypyridine; N-acetylneuraminic acid; N-acetylmannosamine; thymine; and deoxycitidine.

7. The method of claim 1, wherein the predictive computational model is to predict time to delivery and the predictive computational model includes at least two features that are each independently a measurement of one of the following metabolites: 11α-hydroxyprogesterone β-D-glucuronide; 5 β-pregnane-3α, 17-diol-20-one; omega-3 arachidonic acid methyl ester; ubiquinone (Q2); tetrahydrocorticosterone; 5α-pregnane-3,20-dione; 5(Z),8(Z),11(Z)-eicosatrienoic acid methyl ester; 21-hydroxypregnenolone; cortisol; pregnenolone; 19-hydroxytestosterone; progesterone; propionyl-carnitine; androstane-3,17-diol; N-acetyllactosamine; N-(4-chlorophenyl)-4-piperidinamine; 3-acetoxypyridine; N-acetylneuraminic acid; N-acetylmannosamine; thymine; and deoxycitidine.

8. The method of claim 1, wherein the predictive computational model is to predict time to delivery and the predictive computational model includes at least twenty-one features that are each independently a measurement of one of the following metabolites: 11α-hydroxyprogesterone β-D-glucuronide; 5 β-pregnane-3α, 17-diol-20-one; omega-3 arachidonic acid methyl ester; ubiquinone (Q2); tetrahydrocorticosterone; 5α-pregnane-3,20-dione; 5(Z),8(Z),11(Z)-eicosatrienoic acid methyl ester; 21-hydroxypregnenolone; cortisol; pregnenolone; 19-hydroxytestosterone; progesterone; propionyl-carnitine; androstane-3,17-diol; N-acetyllactosamine; N-(4-chlorophenyl)-4-piperidinamine; 3-acetoxypyridine; N-acetylneuraminic acid; N-acetylmannosamine; thymine; and deoxycitidine.

9. The method of claim 1, wherein the model utilizes one or more analyte measurement features, and wherein the one or more analyte measurement features that are utilized is selected based on a contribution to the predictive power of the model.

10. The method of claim 1, wherein the computational model was trained utilizing analyte measurements derived from at least one urine sample collected from each pregnant individual of a cohort of pregnant individuals.

11. The method of claim 10, wherein the at least one urine samples collected from each pregnant individual of the cohort comprise at least one urine sample collected at a routine prenatal visit.

12. The method of claim 10, wherein the computational model was trained utilizing analyte measurements derived from at least three urine samples collected from each pregnant individual of the cohort, wherein each urine sample was collected at three individual timepoints.

13. The method of claim 12, wherein the at least three urine samples collected from each pregnant individual of the cohort comprise at least one urine sample collected in each trimester.

14. The method of claim 12, wherein the at least three urine samples collected from each pregnant individual of the cohort comprise at least one urine sample collected at three individual routine prenatal visits.

15. The method of claim 1, wherein the obtained measurements of one or more analytes are derived from at least two urine samples collected from the individual to be assessed, and wherein the at least two urine samples collected from the individual to be assessed are collected at two individual timepoints.

16. The method of claim 1, wherein the one or more urine samples collected from an individual to be assessed are collected while the individual is fasting.

17. The method of claim 1, wherein the individual has been diagnosed as pregnant.

18. The method of claim 1, wherein the individual has not been diagnosed as pregnant at the time of urine sample collection.

19. The method of claim 1, wherein the measurements of one or more analytes are measured by mass spectrometry, colorimetric analysis, or immunodetection.

20. The method of claim 1 further comprising performing sonography on the individual.

21. The method of claim 1 further comprising treating the individual based on the determined gestational age or time to delivery, wherein the treatment is one of: medication, dietary supplement, caesarian delivery, or surgical procedure.
